(19) United States
(12) Patent Application Publication
(10) Pub. No.: US 2015/0191693 A1
(43) Pub. Date: Jul. 9, 2015

(54) PROCESS FOR MODIFYING A POLYMERIC SURFACE

(71) Applicant: POLYMERS CRC LTD., Notting Hill, Victoria (AU)

(72) Inventors: Thomas Ameringer, Elwood (AU); Laurence Meagher, Brunswick (AU); Helmut Thissen, Rowville (AU); Paul Pasic, Mount Eliza (AU); Katie Styan, Malvern (AU)

(21) Appl. No.: 14/411,793

(22) PCT Filed: Jun. 28, 2013
(86) PCT No.: PCT/AU2013/000710

§ 371 Date: Dec. 29, 2014

(30) Foreign Application Priority Data
Jun. 29, 2012 (AU) 2012902793

(51) Int. Cl. C12N 5/00; B05D 3/06

(52) U.S. Cl. CPC C12N 5/0068; B05D 3/067; C12N 2533/30; C12N 2513/00; C12N 2537/00

(57) ABSTRACT

A process for modifying a polymeric surface, the process comprising; contacting the polymeric surface with a solution comprising at least one ethylenically unsaturated monomer; and exposing the polymeric surface in contact with the solution to ultra-violet light to provide a graft-polymer of the monomer coated on the polymeric surface.

### FIELD

The invention relates to a process for modifying a polymeric surface to provide a grafted polymeric coating.

### BACKGROUND

The modification of a substrate surface by application of a polymer coating is a versatile and efficient means of controlling interfacial properties such as surface energy (e.g. wetting behaviour), permeability, bio-activity, and chemical reactivity. Benefits that may be imparted to a substrate as a consequence of application of a polymer coating include, but are not limited to, chemical sensing ability, wear resistance, gas barrier enhancement, protein resistance, biocompatibility, encouragement of cell growth and differentiation and the ability to selectively bind biomolecules. Methodology for forming such polymer coatings is therefore of great practical benefit.

For example, polymeric materials such as polystyrene have excellent mouldability, transparency and low cost, making them ideal for forming cell culture substrates such as multiwell plates, flasks and microcarrier particles. However, the hydrophobic surface which lacks functional groups limits the ability to control interactions with cells and proteins.

Surface modification may be used to enhance biocompatibility and allow attachment of functional groups which assist in cell binding or selection. Substrate materials modified by grafting of hydrophilic polymer brushes may significantly enhance properties for cell culture applications. A number of surface grafting techniques such as gamma radiation, electron beam and UV-initiated grafting have been examined but there is a need to provide an economic treatment method which provides excellent control over coating properties and ultimately over the biological response.

One approach to forming polymer coatings on a polymeric surface is by using physical or chemical adsorption techniques. Physical adsorption techniques are most commonly used and include dip-coating, drop casting, spin-coating, doctor blade film application, and roll-to roll coating. However, such coatings are prone to delamination upon being exposed to certain chemical and/or physical environments (e.g. organic solvents, temperature variations and/or mechanical abrasion).

An alternative approach to forming polymer coatings involves covalently attaching polymer chains to the surface of the substrate. Unlike the aforementioned adsorption techniques, covalently attaching the polymer chains to the substrate renders the coating less prone to delamination by chemical or physical means. One particular way of covalently attaching polymer chains to a substrate so as to form a polymer coating thereon utilises the so called “grafting to” technique. By this technique, pre-formed polymer chains are covalently attached to the substrate surface. However, due to diffusional and steric limitations at the substrate surface binding sites, this technique is prone to yielding comparatively poor grafting densities. In addition, the coating thickness that can be achieved by this technique is limited.

Polymer chains may also be grafted to the surface of a substrate using the so called “grafting from” technique. Unlike the “grafting to” technique, the “grafting from” technique involves polymerising monomer at the surface of the substrate so as to generate polymer chains “from” the surface. This technique is less prone to the diffusional and steric limitations of the “grafting to” technique and thereby can more readily afford relatively high grafting densities. However, “grafting from” techniques often suffer from being complex and requiring multiple steps. In particular, the surface of a substrate that is to be coated with the graft polymer will generally need to be modified or activated in some way to enable, for example, free radical polymerisation to proceed. Thus, the substrate surface may need to undergo glow or corona discharge pre-treatment to promote the formation of functional groups thereon that can yield the required radical sites. Alternatively, free radical initiator compounds can be immobilised on the substrate surface that is to be grafted. Many “grafting from” techniques are also not capable of effectively and efficiently forming uniform polymer coatings on three dimensional surfaces. Furthermore, the coating thickness can often only be controlled within relatively narrow limits, and as the coating thickness typically is determined by a multitude of factors, control can be difficult to achieve. Accordingly, there remains scope for improving on prior art techniques for forming graft polymer coatings on substrates, or at the very least to provide a useful alternative method for preparing such graft polymer coatings.

The discussion of documents, acts, materials, devices, articles and the like included in this specification is solely for the purpose of providing a context for the present invention. It is not suggested or represented that any or all of these matters formed part of the prior art base or were common general knowledge in the field relevant to the present invention as it existed before the priority date of each claim of this application.

### SUMMARY

We provide a process for modifying a polymeric surface, the process comprising;
contacting the polymeric surface with a solution comprising at least one ethylenically unsaturated monomer; and
exposing the polymeric surface in contact with the solution to ultraviolet light to provide a graft-polymer of the monomer as a coating on the polymeric surface.

It is preferred that the polymeric surface and solution are free of initiators.

In one set of embodiments the solution of ethylenically unsaturated monomer is an aqueous solution optionally comprising one or more water miscible solvents. It is thus generally preferred in this embodiment that the ethylenically unsaturated monomer is at least sparingly water soluble and is more preferably water soluble.

The exposure of the surface to ultraviolet light will generally be pulsed or intermittent exposure to UV radiation. We have found that the graft architecture fromethylenically unsaturated monomers and in particular water soluble ethylenically unsaturated monomers on polymeric surfaces is significantly improved when using UV grafting if the polymeric surface is exposed intermittently to ultraviolet light while in contact with the solution of the ethylenically unsaturated monomer.

The invention may involve pulsed or intermittently exposing the polymeric surface to ultraviolet light. Intermittently exposing the polymeric surface to ultraviolet light is particularly preferred and has been found to provide significant advantages in architecture of the graft polymer formed from the ethylenically unsaturated monomer. The architecture provided by intermittently exposing the polymeric surface provides improved swelling of the coating in aqueous environments when compared with corresponding graft polymer coating prepared by continuous exposure.

Without wishing to be bound by theory we believe the intermittently exposing the polymeric surface to ultraviolet light creates differences in architecture between polymer layers formed during periods of exposure to ultraviolet light and periods without exposure to ultraviolet light.

The term intermittent exposure as used herein refers to periods of UV light exposure (on-periods) of duration of at least about 0.5 seconds, more preferably at least about 1 second, more preferably at least about 2 seconds. The duration of exposure (on-period) may be up to about 3 minutes, more preferably up to about 60 seconds and still more preferably up to about 45 seconds. The period between exposures (the off-period) may be of duration up to about 60 minutes, more preferably up to about 30 minutes, more preferably still up to about 10 minute such as up to 5 minutes, up to 2 minutes and up to 1 minute. The period between exposures (the off-period) may be of duration at least about 5 seconds, preferably at least about 10 seconds, more preferably at least about 15 seconds, more preferably at least about 20 seconds, more preferably still at least about 25 seconds.

In one set of embodiments the process of intermittently exposing the polymeric surface to ultraviolet light involves periods of UV exposure in the range of from 0.5 seconds to three minutes with the time between exposures being in the range of from five seconds to 60 minutes. The number of exposures to ultraviolet light is generally at least three exposures. In a further set of embodiments the process involves intermittently exposing the polymeric surface comprises subjecting the surface while in contact with the aqueous solution to in the range of from five to one hundred exposures to ultraviolet light lasting in the range of from 0.5 seconds to five minutes such as 0.5 seconds to 3 minutes with a time gap between exposures being in the range of from one second to 60 minutes such as 5 seconds to 60 minutes or 10 seconds to five minutes.

The term pulsed exposure refers to periods of exposure (on-periods) less than 0.05 s with intervals between exposure of less than 0.05 s. Pulse widths (ON plus OFF period) of 10 μs to 300 μs may be provided using industrial flash lamp systems.

By a “graft” polymer is meant that the polymer chains are covalently coupled to at least the surface of the polymeric surface. The grafted polymer chains may be homopolymer chains or copolymer chains. By the graft polymer being a “coating” is meant that a plurality of polymer chains is covalently coupled to the surface of the polymeric surface so as to collectively form a layer of the graft polymer. The graft polymer chains may be crosslinked. The coating will generally modify the surface properties of the grafted region of the polymeric surface.

Throughout the description and the claims of this specification the word “comprise” and variations of the word, such as “comprising” and “comprises” is not intended to exclude other additives, components, integers or steps.

### DETAILED DESCRIPTION

A polymeric surface is provided upon which a polymeric coating is to be grafted by the method of the present invention. Examples of suitable polymeric surfaces include, but are not limited to surfaces comprising one or more polymers selected from the group consisting of, polyolefins such as polyethylene and polypropylene, polyisobutylene and ethylene-alphaolefin copolymers, silicone polymers such as polydimethylsiloxane; acrylic homopolymers and copolymers, such as polyacrylate, polymethylmethacrylate, polyethylacrylate; vinyl halide homopolymers and copolymers, such as polyvinyl chloride; fluoropolymers such as fluorinated ethylene-propylene; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; natural and synthetic rubbers, including butadienestyrene copolymers, polyisoprene, polybutadiene, butadiene-acrylonitrile copolymers, polychloroprene rubbers, polyisobutylene rubber, ethylenepropylenediene rubbers, isobutylene-isoprene copolymers and polyurethane rubbers; polyamides such as Nylon 66 and polycaprolactam; polyesters such as polyethylene terephthalate, alkyd resins; phenol-formaldehyde resins; urea-formaldehyde resins, melamine-formaldehyde resins; polycarbonates; polyoxyalkylenes such as polyoxyethylene, polyoxypropylene and their block copolymers; polyimides; polyethers; epoxy resins, polyurethanes; wool; cotton; silk; rayon; rayon-triacetate; cellulose, cellulose acetate, cellulose butyrate; cellulose acetate butyrate; cellophane; cellulose nitrate; cellulose propionate; cellulose ethers; carboxymethyl cellulose; proteins, polypeptides; and polysaccharides.

In some embodiments, the polymeric surface is one comprised of only saturated carbon-carbon bonds and is free of double or triple carbon-carbon bonds. In such polymeric surfaces, the minimum bond energy present is typically greater than in polymeric surfaces including unsaturated carbon-carbon bonds. Examples of such polymeric surfaces may be selected from the group including polyolefins such as polyethylene and polypropylene, polyisobutylene and ethylene-alphaolefin copolymers and polyvinyl aromatics, such as polystyrene, styrene copolymers, poly-isoprene, synthetic polyisoprene, polybutadiene, polychloroprene rubbers, polyisobutylene rubber, ethylene-propylenediene rubbers and isobutylene-isoprene copolymers

In accordance with the method of the invention, the polymeric surface and solution of ethylenically unsaturated monomer are substantially free of radical initiator. By being “substantially free of radical initiator” is meant a radical initiator per se is not included or introduced to the polymeric surface or solution of ethylenically unsaturated monomer. For example, the polymerisation is to be performed in the absence of radical initiators.

Those skilled in the art will appreciate that some monomers may, upon being exposed to UV radiation, decompose to afford a radical species. For avoidance of any doubt, monomers that are polymerised to form the graft polymer coating are not intended to be embraced within the definition of a radical initiator. The expression “radical initiator” is intended to mean compounds that are used primarily for the purpose of generating free radicals and includes photoinitiators such as benzophenone and acetophenone derivatives such as diethoxy acetophenone and other radical initiators such as azo initiators and peroxides.

The surface may have been treated by a process such as corona discharge. Such treatment processes are sometimes used in manufacture of polymeric articles such as film or cell culture plates which may be modified using the process. Generally the effect of corona discharge is relatively short lived so that after storage the surface is deactivated.

The polymeric surface to be modified may constitute all or only part of an article to which the method of the invention is applied. For example, the graft polymeric coating may be formed on at least part of a polymeric surface. In the case where the article is formed of polymer the bulk of the polymer may remain unmodified so that the mechanical properties of the article are maintained. Alternatively, the polymeric surface to be modified may itself present as a coating on a substrate. The substrate may be polymeric, or alternatively may be a non-polymeric substrate such as a glass, ceramic or metal substrate.

In some embodiments, the polymeric surface is present on or part of an article that is used for the culture of cells. The cell culture device may be in a range of structural forms known in the art. Such structural forms include culture plates such as microtitre or microwell plates including comprising a multiplicity of wells such as 6, 12, 24, 48 96, 1536 or more wells and cell culture flasks. The substrate may also be in the form of carrier particles such as microcarrier particles. In these embodiments, it is preferred that the substrate surface to be modified is transparent.

Without wishing to be limited by theory, it is believed that polymerisation of the ethylenically unsaturated monomer in accordance with the invention occurs predominantly at the polymeric surface such that the polymer is grafted from that surface. By polymer being grafted predominantly from the polymeric surface it is in turn believed that improved control over at least coating efficiency can be attained.

Due to its lack of complexity, the method in accordance with the invention can advantageously be performed with relatively low operating and capital costs. Furthermore, the method of the invention has been found to be particularly effective at forming in a controlled manner graft polymer coatings with a relatively wide range of thicknesses on the surface of substrates having varied shapes and sizes, and in particular on substrates that present a three dimensional surface. Great variability in polymeric surface and monomer is also possible.

The method of the invention comprises contacting the polymer surface with a solution comprising at least one ethylenically unsaturated monomer. The solution may be aqueous, partially aqueous, or non-aqueous. In some embodiments it is preferable that the solution is at least partially aqueous and further comprises at least one water miscible organic solvent. In other embodiments, it is preferable that the solution is aqueous. The choice of the most suitable solution will be dependent on the polymeric surface, the ethylenically unsaturated monomer, and the intended application of the polymeric coating.

The solution may comprise one or more solvents such as water, water miscible solvents or mixtures of two or more thereof. Examples of water miscible solvents include dimethoxysulfoxide (DMSO), dimethylformamide (DMF), acetonitrile, acetone and alcohols such as ethanol and isopropanol. Where one or more water miscible solvents are present in the ethylenically unsaturated monomer solution, they will be selected so as to not adversely affect the polymerisation reaction, the polymeric surface, or the resulting graft polymer coating.

The method of the invention is advantageously performed using an environmentally friendly aqueous solution of the ethylenically unsaturated monomer in contrast with conventional organic solvent based polymerisation reactions. Furthermore, the aqueous solution is compatible with a broader range of polymeric surfaces compared with that available when organic solvent based reaction mediums are used. The use of an aqueous solution also provides a product which is readily prepared for biological applications such as cell culture or use as biomedical surfaces.

In a preferred embodiment, the polymeric coating is employed as cell cultureware and the solution is aqueous.

The concentration of monomer present in the solution will vary depending upon the nature of the polymer coating that is to be formed. For example, the concentration of the one or more ethylenically unsaturated monomers may be adjusted to tailor the thickness of the polymer coating. Those skilled in the art will be able to determine the required concentration of ethylenically unsaturated monomer for a given polymerisation. Generally, the concentration of the one or more ethylenically unsaturated monomers in the solution will fall within the range of about 0.1% (w/v) to about 25% (w/v).

Other additives that may be present in the aqueous reaction medium include polymerisation inhibitors. These are often present in commercially available monomers to extend their shelf life. The fact that these inhibitors may be present is an advantageous feature of the invention as monomers can be used without the need to remove the inhibitor prior to polymerisation.

The polymerisation in accordance with the invention is preferably conducted in a substantially oxygen free environment. In other words, the polymerisation is to proceed under substantially oxygen free conditions. This may be achieved using techniques well known to those skilled in the art. For example, the monomer solution and any head space above the solution may be purged with an inert gas such as nitrogen or argon. The presence of oxygen can interfere with the efficiency of the polymerisation process. The fact that the reaction proceeds, albeit slowly, despite the presence of oxygen is an advantageous feature of the invention as it can be used without the need to fully remove oxygen prior to polymerisation.

Further, the monomer solution is generally deoxygenated prior to irradiation to reduce scavenging of radicals. Suitable deoxygenation methods include methods known to those skilled in the art such as bubbling the inert gas through the monomer solution or freeze-thaw-pump cycles.

Examples of ethylenically unsaturated monomers that may be used in accordance with the invention include, but are not limited to, methyl (meth)acrylate, ethyl (meth)acrylate, ethyl-3,3-dimethyl (meth)acrylate, butyl (meth)acrylate, isobutyl (meth)acrylate, isobutyl(meth)acrylate, tert-butyl (meth)acrylate, 2-ethylhexyl (meth)acrylate, isobornyl (meth)acrylate, (meth)acrylic acid, hydroxypropyl (meth)acrylate, hydroxybutyl (meth)acrylate, (meth)acrylamide, 2-hydroxyethyl (meth)acrylate, N-methyl (meth)acrylamide, dimethylaminoethyl (meth)acrylate, itaconic acid, 2-carboxyethyl acrylate, styrene, p-styrene carboxylic acids, p-styrene sulfonic acids, vinyl sulfonic acid, vinyl phosphonic acid, ethacrylic acid, alpha-chloroacrylic acid, crotonic acid, fumaric acid, citraconic acid, mesaconic acid, maleic acid, glycidyl (meth)acrylate, hydroxyethyl (meth)acrylate succinate, 2-(meth)acrylamido-2-methyl-1-propanesulfonic acid, 2-sulfoethyl(meth)acrylate, 3-sulfopropyl (meth)acrylate, mono-2-[(meth)acryloyloxy]ethyl succinate, hydroxypropyl (meth)acrylate, N-ethyl (meth)acrylamide, N,N-dimethyl(meth)acrylamide, N,N-diethyl (meth)acrylamide, N-isopropyl(meth)acrylamide, N-(hydroxymethyl) (meth)acrylamide, N-(2-hydroxyethyl) (meth)acrylamide, N-(2-hydroxypropyl) (meth)acrylamide, N-methylol (meth)acrylamide, N-vinylformamide, N-vinylacetamide, N-vinyl-N-methylacetamide, N-(n-propyl)acrylamide, N-(n-butyl)(meth)acrylamide, N-tert-butyl (meth)acrylamide, cyclohexyl (meth)acrylamide, N-(3-aminopropyl) (meth)acrylamide, 2-aminoethyl (meth)acrylate, N-[3-(dimethylamino)propyl] (meth)acrylamide, N-(meth)acryloyl tris(hydroxymethyl) aminoethane, N-(meth)acryloyl tris(hydroxymethyl) aminoethane, diacetone (meth)acrylamide, 2-(meth)acryloyloxy ethyl acetoacetate, [3-(methacryloylamino)propyl]trimethylammonium chloride, [3-(methacryloyloxy)ethyl]-trimethylammonium chloride, [2-(methacryloyloxy)ethyl]dimethyl-(3-sulfopropyl)ammonium hydroxide, [3-(methacryloylamino)propyl]dimethyl(3-sulfopropyl)ammonium hydroxide, poly(ethylene glycol) (meth)acrylate, poly(ethylene glycol) methyl ether (meth)acrylate, poly(propylene glycol) (meth)acrylate, poly(propylene glycol) methyl ether (meth)acrylate, propargyl (meth)acrylate, 4-(meth)acryloylmorpholine, N-vinyl-2-pyrrolidone, glycerol mono(meth)acrylate, glycosyloxyethyl (meth)acrylate, vinyl methyl sulphone, vinyl acetate, 2-(meth)acryloxyethyl glucoside, ethylene glycol (meth)acrylate phosphate, ethylenically unsaturated mono-, di-, tri and polysaccharide(s) where the saccharide moiety is net neutral, zwitterionic monomers such as 3-((2-(meth)acryloyloxy)ethyl)dimethylammonio) propane-l-sulfonate, 2-((meth)acryloyloxy)ethyl 2-(trimethylammonio)ethyl phosphate, 2-methacryloyloxyethyl phosphorylcholine and combinations thereof.

Examples of ethylenically unsaturated monomers that may be used in accordance with the invention also include “ligands” comprising one or more ethylenically unsaturated groups. The term “ligand” used herein is intended to take its common meaning within the art being a moiety that can bind a specific biomolecule, for example a biomolecule expressed on the surface of a cell, in the presence of a multitude of other biomolecules.

The polymer grafted to the polymeric surface may be a homopolymer or copolymer, depending on the ethylenically unsaturated monomers employed. The polymer may further be charged or neutral, and may belong to a class of polymer selected from the group consisting of carboxylic acid polymers, sulfonic acid polymers, amino polymers, zwitterionic polymers, neutral hydrophilic polymers and hydrophobic polymers.

In some embodiments, the solution comprises a single type of ethylenically unsaturated monomer. The ethylenically unsaturated monomer may be selected from any one of those described herein.

In some embodiments, the solution comprises at least 2 ethylenically unsaturated monomers.

Those skilled in the art will appreciate that factors such as temperature, pH and/or the presence or absence of water miscible co-solvent(s) may alter the solubility of a given monomer in a given aqueous monomer solution. Such factors can therefore be conveniently used to promote the solubility of monomer in an aqueous solution.

In some embodiments, the ethylenically unsaturated monomer is at least sparingly soluble in water, and more preferably soluble in water. The term sparingly soluble refers to a solubility of one gram monomer to 100 mL solvent and soluble refers to at least 1 gram monomer to 10 mL water at 20° C.

Examples of preferred ethylenically unsaturated monomers which are water soluble include acrylic acid, methacrylic acid, 2-carboxyethyl acrylate, hydroxyethyl (meth)acrylate succinate, acrylamide, methacrylamide, N-alkyl (meth)acrylam ides (such as N-isopropyl acrylamide), N,N-dimethyl (meth)acrylamide, N-(3-aminopropyl) (meth)acrylamide, 2-aminoethyl (meth)acrylate, dimethylaminoethyl (meth)acrylate, N-vinyl-2-pyrrolidone, 2-hydroxyethyl (meth)acrylate, N-(2-hydroxypropyl) (meth)acrylamide, 2-methacryloyloxyethyl phosphorylcholine, 3-sulfopropyl (meth)acrylate, [3-(methacryloylamino)propyl]trimethylammonium chloride, [3-(methacryloyloxy)ethyl]-trimethylammonium chloride, poly(ethylene glycol) (meth)acrylate and methoxy poly(ethylene glycol) (meth)acrylate. Particularly preferred water soluble monomers include acrylic acid, 2-carboxyethyl acrylate, acrylamide, N-isopropyl acrylamide, N-(2-hydroxypropyl) methacrylamide, 2-methacryloyloxyethyl phosphorylcholine, poly(ethylene glycol) (meth)acrylate and methoxy poly(ethylene glycol) (meth)acrylate.

In some embodiments, the solution comprises 2 monomers. A first monomer preferably includes a carboxylic acid functional group, and the resulting polymeric coating immobilised on the polymeric surface then comprises carboxylic acid groups. Preferably, the first monomer is acrylic acid, methacrylic acid or 2-carboxyethyl acrylate. A second monomer is preferably providing low biofouling properties such that a polymeric coating formed using the method of the present invention is non-adhesive to mammalian cells in serum-containing culture medium. Preferably, the second monomer is acrylamide, poly(ethylene glycol) (meth)acrylate, methoxy poly(ethylene glycol) (meth)acrylate, N-(2-hydroxypropyl) methacrylamide, 2-methacryloyloxyethyl phosphorylcholine or the like as would be known in the art. In a particular preferred embodiment, the first monomer is acrylic acid and the second monomer is acrylamide. In these embodiments, the molar ratio of the first to second monomer in the solution can be at least 1:99 such as at least 5:95, at least 10:90 or at least 20:80 and up to 90:10 molar such as up to 80:20 whilst retaining low biofouling. More preferably, the ratio is 40:60 to 80:20 such as 80:20, 70:30, 60:40, 50:50 or 40:60. In particularly preferred embodiments, the molar ratio is 40:60.

Having contacted the polymeric surface with the solution of ethylenically unsaturated monomer, that surface is then exposed to UV radiation so as to generate radical species thereon. Those skilled in the art will appreciate that UV radiation is typically defined as electromagnetic radiation having a wavelength shorter than visible light, but longer than X-rays, and therefore has a wavelength within the range of about 10 nm to about 400 nm. There is no particular limitation concerning the wavelength of UV radiation that may be used in accordance with the invention provided that it can generate free radicals on the polymeric surface. Generally, the wavelength of UV radiation used will fall within the range of about 200 nm to about 400 nm. The ultraviolet light used in the process is preferably up to 400 nm wavelength. The ultraviolet light used in the process is more preferably up to 300 nm wavelength. A range of suitable sources of ultraviolet light may be used and high intensity microwave electrode less bulb sources are particularly preferred.

Provided that the free radicals can be generated on the polymeric surface, and that the surface is not adversely affected, there is also no particular limitation as to the intensity of the UV radiation that can be used. Generally, UV sources with an output of at least up to about 200 W/cm² can be used.

In a preferred set of embodiments the aqueous solution is washed from the surface with water after exposure to irradiation.

The polymeric surface in contact with the solution is exposed to a period of irradiation. One period of irradiation may be considered an on-period, during which the polymeric surface is exposed to irradiation, followed by an off-period, during which the polymeric surface is not exposed to irradiation. For continuous exposure there is one period, with the on-period being of definite length and the off-period being of indefinite length. The present invention relates to either pulsed or intermittent irradiation, both of which have at least 2 periods of exposure.

For pulsed irradiation the periods of exposure (on-periods) are generally less than 0.05 s with intervals between exposures (off periods) of less than 0.05 s.

In preferred embodiments, the polymeric surface in contact with the solution is exposed to intermittent irradiation. By definition, the intermittent irradiation includes at least 2 periods of exposure. The total number of periods of exposure may be selected based on the desired properties of the polymeric coating. For instance, in applications requiring a thicker polymeric coating, or for polymer surfaces more susceptible to UV degradation, the number of periods of exposure can be increased.

In preferred embodiments, a thick coating is required to render the underlying polymeric surface ‘invisible’ to mammalian cells cultured on the polymeric coating. In these embodiments, up to 100 periods of exposure may be employed.

The optimum duration of the periods of irradiation will also depend on the nature of the polymeric surface on to which grafting is to occur and the intensity of irradiation. In some instances extended periods of irradiation may lead to deterioration and/or deformation of the polymeric surface. For example in the case of surfaces formed of polystyrene exposure times not in excess of about 45 seconds are preferred, and more preferably less than about 30 seconds. A person skilled in the art will be able to determine suitable combinations of UV intensity and periods of exposure having regard to the nature of the surface and graft monomer composition, and the teaching herein.

The absolute and relative durations and powers of the exposure to ultraviolet light (on-period) and between exposures to ultraviolet light (off-periods) should be selected so as to be suitable for the polymeric surface and to provide a polymeric coating of desired properties. Particularly important properties in some embodiments are the polymeric coating thickness and elastic modulus. For example, it is hypothesised that relatively greater on-period exposure will result in denser and thinner polymeric coatings, while relatively greater off-period exposure will result in softer, thicker, more swellable polymeric coatings.

For intermittent irradiation, the on-period should be such that the structural integrity of the article, polymeric surface, and/or developing polymeric coating is maintained. During the on-period it is hypothesised that the exposure to irradiation causes bond scission, relatively highly cross-linked polymer growth, and substantial heat generation. Depending on the chemistry of the components involved in the process, the on-period could lead to detrimental effects. As such, the duration and power of the on-period should be chosen to avoid, or at least mitigate to suitable levels, these detrimental effects. The on-period should also be selected to be sufficient to initiate polymerisation.

For example, the on-period may be of duration of at least about 0.5 seconds, more preferably at least about 1 second, more preferably still at least about 2 seconds. The on-period may be of duration up to 60 seconds, more preferably up to about 45 second, more preferably still up to about 30 seconds. In preferred embodiments, where the polymeric surface comprises polystyrene, the on-period is of duration from about 1 second to about 45 seconds such as about 5 seconds to about 45 seconds.

In a preferred set of embodiments the intermittent irradiation comprises on-periods in the range of from 1 second to 15 seconds and off-periods in the range of from 1 second to 60 seconds.

UV light sources with an output of up to about 200 W/cm² have been used.

For intermittent irradiation, the off-period should be such that the final polymeric coating is of sufficient properties. During the off-period it is hypothesised that even in the absence of exposure to irradiation, polymer growth continues in a ‘growth-from’ non-cross-linked mode, and any heat generated during the on-period can dissipate to some degree. The duration of the off-period should be chosen giving consideration to these factors. At a certain point, polymer chain growth in this off-period will cease, therefore there is no anticipated benefit from an off-period longer than this, but this period will be dependent on the polymer surface, the monomer solvent, and the monomer, at least.

For example, the off-period may be of duration less than about 5 minutes, more preferably less than about 3 minutes, more preferably still less than about 2 minute. The off-period may be of duration more than about 10 seconds, more preferably more than about 20 seconds, more preferably still more than about 30 seconds, more preferably still more than about 45 seconds. In preferred embodiments, where the polymeric surface comprises polystyrene, the off-period is of duration from about 20 seconds to about 60 seconds such as 20 seconds to about 50 seconds.

Without wishing to be limited by theory, it is believed that exposing the polymeric surface to UV radiation causes bonds that make up the molecular structure of that surface to undergo cleavage so as to generate radical species. The generated radical species can then promote free radical polymerisation of the one or more ethylenically unsaturated monomers present within the monomer solution. Polymerisation of the monomers in this way is believed to provide for polymer chains being grafted from the polymeric surface.

Without wishing to be limited by theory, it is believed that exposing the polymeric surface to UV radiation may also, or alternatively, cause the ethylenically unsaturated bonds of the monomer to undergo cleavage so as to generate radical species. The generated radical species can then via hydrogen abstraction from the polymer surface promote free radical polymerisation of the one or more ethylenically unsaturated monomers present within the monomer solution. Polymerisation of the monomers in this way is believed to provide for polymer chains being grafted from the polymeric surface.

Without wishing to be limited by theory, it is also believed that carbon based radicals are generated on the polymeric surface, and it is these radicals that are responsible for promoting polymerisation of the one or more ethylenically unsaturated monomers. Where the polymeric surface comprises a carbon based polymer, the formation of such carbon based radicals is believed to be facilitated when the carbon based polymer used comprises a carbon-carbon polymer backbone.

The nature of the graft polymer coating formed on the polymeric surface can advantageously be varied to suit the intended application of the resulting product.

One advantage of the method is that it can afford a substantially uniform and continuous graft polymer coating on the polymeric surface, be it a two dimensional or a three dimensional surface. By the graft polymer coating being “substantially uniform and continuous” is meant that it presents over the desired region of the polymeric surface and has an integral coating having a relatively constant thickness. Having said this, the graft polymer coating may of course present as a discontinuous coating on the polymeric surface in that it may be formed on only a part or parts of that surface. In that case, the graft polymer will nevertheless form a substantially uniform and continuous coating on those parts of the polymeric surface. For example, it may be desirable to form a particular pattern or array of the graft polymer coating on the polymeric surface. This can be achieved, for example, by passing the UV radiation through a suitable mask that limits regions of the polymeric surface to UV radiation exposure.

The thickness of the graft polymer coating applied to the polymeric surface can be varied by adjusting parameters of the method well known to those skilled in the art. For example, the thickness of the graft polymer coating may be increased by increasing the concentration of ethylenically unsaturated monomer present within the solution by increasing the time and/or intensity of UV radiation exposure. Coatings having a gradient thickness may also be produced by having a gradient mask between the UV source and the polymeric surface.

As used herein, the term “biomolecule” is intended to mean molecules that are produced by an organism, tissue or cell. Biomolecules include, but are not limited to, peptides, oligopeptides, polypeptides, proteins, nucleic acids, nucleotides, carbohydrates and lipids.

As an alternative to, or in combination with, forming a graft polymer coating that resists biomolecule adsorption and/or biofouling, it may be desirable to include as part of the graft polymer coating a ligand for binding with a specific biomolecule such as a specific biomolecule in solution or a specific biomolecule expressed on the surface of a cell.

By this approach, specific biomolecules per se or cells can be targeted for attachment to the graft polymer coating in applications such as assays and cell culture.

The graft polymer coating may be provided with such a ligand by any suitable means. For example, the ligand may be covalently bound to or comprise one or more of the ethylenically unsaturated monomers that are polymerised to form the graft polymer coating.

Alternatively, the graft polymer coating may be modified after it is formed so as to covalently couple the ligand to the surface of the graft polymer coating. In that case, one or more ethylenically unsaturated monomers that are polymerised to form the graft polymer coating may be provided with a functional group that can be used subsequent to the formation of the graft polymer coating to facilitate the covalent attachment of the ligand to the graft polymer coating.

As used herein, the term “cell” refers to a live or dead cell, multicellular, tissue or cellular fragments, cell membrane, liposomal preparation or sub-organelle such as mitochondria, ribosome or nucleus. The term “cell” is also intended to include adherent and non-adherent cell types.

The cells may be eukaryotic or prokaryotic cells.

Eukaryotic cells include those derived from animals/humans, plants, fungi, and protists.

Prokaryotic cells include those derived unicellular microorganisms such as bacteria and archaea.

The term “cell” is also intended to include stem cells. In animals/humans most adult stem cells are lineage-restricted (multipotent) and are generally referred to by their tissue origin. For example, embryonic stem cells, mesenchymal stem cells, adipose-derived stem cells, endothelial stem cells, hematopoietc stem cells, neural stem cells, epithelial stem cells and skin stem cells etc.

We have found that the coatings made using continuous UV irradiation was significantly thinner than coatings prepared using equivalent intermittent UV irradiation conditions. Furthermore we found that the coating made using intermittent irradiation with delay between irradiation events was significantly thicker than the coating made with intermittent UV irradiation.

Analysis of the swelling ratio of the three coatings (see Table 12) indicated that the coatings made with intermittent UV irradiation were able to swell much more that the coating made using continuous UV irradiation when hydrated with PBS solution. Without wishing to be bound by theory we believe this difference is most likely due to the degree of cross-linking within the coatings. Continuous UV irradiation will lead to four processes; (i) free radical formation, (ii) chain scission, (iii) cross-linking reactions, and (iv) polymer chain growth. These four processes will also occur for graft polymer coatings prepared using intermittent UV irradiation but the relative balance of the four processes will most likely be different. Assuming that the free radical formation is equal in both cases, intermittent UV irradiation should lead to more polymer growth when the sample is not being irradiated with UV than in the continuous case and less cross-linking within the coatings. This hypothesis is borne out by both the dry and hydrated thickness which were both greater in the case of samples prepared with intermittent UV irradiation. The reduced swelling ratio obtained for the samples prepared using continuous UV irradiation suggest that there was a higher degree of cross-linking within the coatings. Thicker coatings such as those prepared using intermittent UV irradiation, with an equal degree of cross-linking would have a very similar swelling ratio. The influence of additional time with no UV irradiation (intermittent+delay) was to increase the polymer graft layer thickness, again suggesting that when the sample was not being irradiated, more polymer growth was occurring than for shorter non-irradiation times (intermittent) and much more polymer chain growth was occurring than in the continuous UV irradiation condition.

The invention will now be described with reference to the following examples. It is to be understood that the examples are provided by way of illustration of the invention and that they are in no way limiting to the scope of the invention.

### BRIEF DESCRIPTION OF DRAWINGS

The Examples are described with reference to the attached drawings.

In the drawings:

FIG. 1A and FIG. 1B show representative high resolution XPS C 1s spectra obtained on tissue culture polystyrene plates (Nunclon™ Δ, Nunc) before (FIG. 1A) and after (FIG. 1B) UV graft polymerisation of AAM as described in Example 1.

FIG. 2A and FIG. 2B show phase contrast images of HeLa cell attachment after 20 hours on PS (Nunclon™ Δ) substrates after UV grafting of AAM (FIG. 2A) in comparison to cell attachment on a PS (Nunclon™ Δ) control surface (FIG. 2B) as described in Example 3.

FIG. 3 shows a representative high resolution XPS C 1s spectra obtained on UV graft polymers obtained from mixtures of AA and AAM monomer solutions. The numbers (insert) refer to the percentage of AA in the monomer mixture as described in Example 4.

FIG. 4A and FIG. 4B show phase contrast images (10× objective) of L929 mouse fibroblast cell attachment on NIPAM UV graft polymer coated substrates at (FIG. 4A) 37° C. and (FIG. 4B) after 30 min incubation at 20° C. as described in Example 6, Part B

FIG. 5A and FIG. 5B show phase contrast images (10× objective) of human MSC attachment on NIPAM UV graft polymer coated substrates at (FIG. 5A) 37° C. and (FIG. 5B) after 30 min incubation at 20° C. as described in Example 6, Part C.

FIG. 6A and FIG. 6B show representative phase contrast images (10× objective) of L929 cell attachment on MicroHex™ microcarrier particles coated with a NIPAM graft polymer coating at (FIG. 6A) 37° C. and (FIG. 6B) after 30 min incubation at 20° C. as described in Example 7, Part B.

FIG. 7A and FIG. 7B show representative images of L929 cell attachment on 96 well substrates coated with (FIG. 7A) a 10% AA UV graft copolymer and (FIG. 7B) the same surface after covalent immobilisation of c(RGDfK) peptide as described in Example 9, Part C.

FIG. 8A and FIG. 8B show representative images of L929 cell attachment on MicroHex™ substrates coated with (FIG. 8A) a 10% AA UV graft copolymer and (FIG. 8B) the same surface after covalent immobilisation of c(RGDfK) peptide as described in Example 10, Part C.

FIG. 9 is a graph showing the cell attachment in response to coatings prepared using two different UV methods as a function of the composition of the polymeric coating in accordance with the description in Example 12, Part C.

FIG. 10 is a graph comparing different cell types in response to copolymer coatings based on acrylic acid (AA) and acrylamide (AAM) which were produced by the initiator-free UV based coating method using intermittent UV as described in Example 12 Part C.

FIG. 11 is a graph showing the elemental ratio obtained from XPS analysis of graft polymer coatings with the number of UV passes as explained in Example 14 Part B.

FIG. 12 is a graph showing the change in thickness of 40% AA-co-AAM layer in nanometres with the number of UV passes.

FIG. 13: A, B and C are high resolution C 1s spectra with various percentages of UVA, UVB and UVC as described in Example 15, Part B.

### EXAMPLES

### Example 1

### UV Graft Polymerisation from Tissue Culture Polystyrene Substrates

96 well tissue culture polystyrene (TCPS) plates (Nunclon™ Δ, Nunc) were used as received and placed into a glove box. In the glove box (under a nitrogen atmosphere containing <0.2% oxygen), 250 mg of acrylamide (AAM), poly(ethylene glycol) methacrylate (PEGMA-OH), methoxy poly(ethylene glycol) methacrylate (PEGMA-OMe) or N-isopropylacrylamide (NIPAM) were dissolved in 5 cm³ Milli-Q™ water and the solution purged for 10 min with nitrogen to remove residual oxygen. Each well of the 96 well plates was then filled with 0.15 cm³ of the monomer solution. After vacuum sealing of the plates into polymer bags (Sunbeam FoodSaver) in the glove box, the plates were placed onto a conveyor belt which ran underneath a UV lamp (λ˜200-450 nm, maximum intensity 360-390 nm, length of lamp 15 cm, output 1.8 kW, FUSION systems). The average belt speed was kept at 1.8 m·min⁻¹ to give an irradiation time of approximately 4 sec per pass. The vacuum sealed plates were exposed to UV irradiation during 30 passes under the UV lamp. In the case of NIPAM graft polymer coatings, plates were exposed during 20 passes under the UV lamp. The orientation of the plates was rotated by 90 degrees after each pass. The wells were then thoroughly washed with Milli-Q™ water using a plate washer (Thermo Wellwash 4 MK 2) and finally air dried. For XPS analysis, the bottom of the wells of interest was removed using a cutting tool.

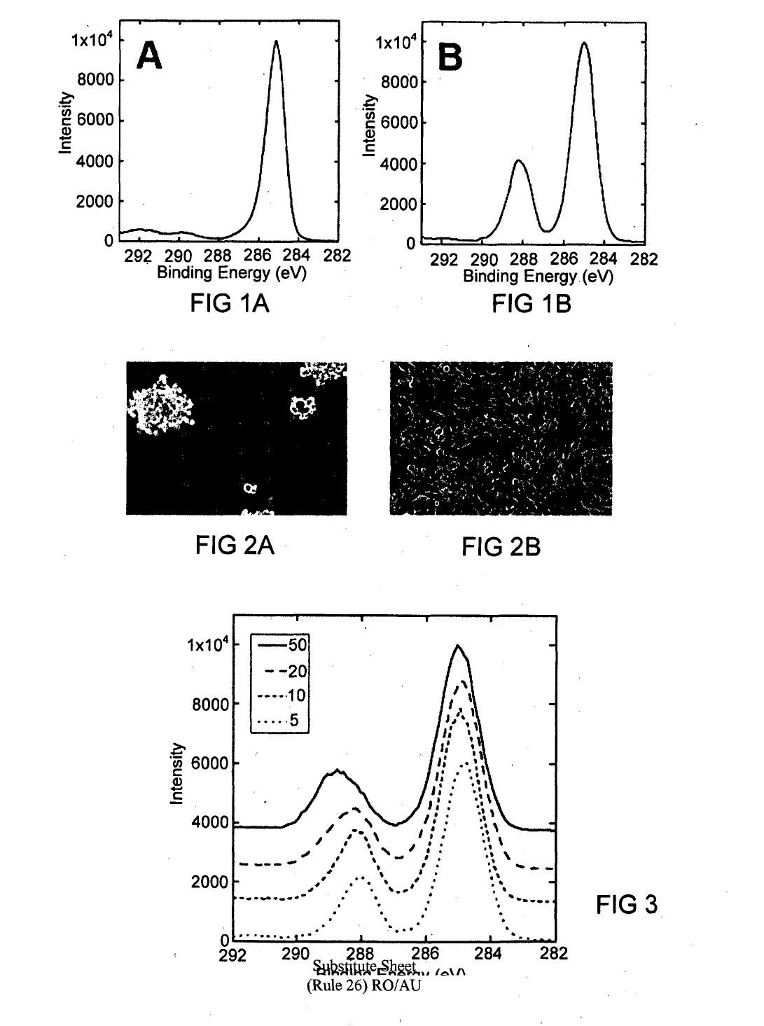

Presented in Table 1 are the elemental ratios, obtained by XPS analysis, before and after UV graft polymerisation on the tissue culture polystyrene plates. The significant changes in the O/C ratio observed for each of the monomers and the changes observed for the N/C ratio after graft polymerisation with the AAM and NIPAM monomer solutions compared to that obtained for the TCPS substrate polymer indicate successful graft polymerisation for each of the monomers. In addition, presented in FIG. 1A and FIG. 1B are the XPS high resolution C 1s spectra obtained from a tissue culture polystyrene plate before and after UV graft polymerisation of AAM. Again the significant differences between these spectra demonstrate the successful grafting of the AAM monomer. The high resolution XPS spectrum presented in FIG. 1AA contains a dominant peak at 285.0-285.5 eV, corresponding to the neutral carbon species C1 and C2 (C—C/C—H) and two smaller peaks at higher binding energy, corresponding to the C5 component (O—C═O) due to oxidised species originating from the surface treatment process and the C6 component corresponding to the aromatic carbon shake-up peak at approximately 292.0 eV. In comparison, the spectrum in FIG. 1B contains a peak at 285.0-285.5 eV, corresponding to the aliphatic carbon species C1 and C2 (C—C/C—H) and a peak at higher binding energy corresponding to the amide species C4 (O═C—N). The complete attenuation of the aromatic shake-up peak in FIG. 1B also suggests a coating thickness of more than 10 nm (XPS sampling depth).

TABLE 1
Elemental ratios calculated from XPS survey spectra obtained on tissue
culture polystyrene plates (Nunclon ™ Δ, Nunc) before and
after UV graft polymerisation.

| Sample | O/C | N/C |
|---|---|---|
| PS (Nunclon ™ Δ) | 0.079 | 0.000 |
| PS-AAM | 0.258 | 0.273 |
| PS-PEGMA-OH | 0.464 | 0.000 |
| PS-PEGMA-OMe | 0.416 | 0.000 |
| PS-NIPAM | 0.131 | 0.095 |

### Example 2

### UV Graft Polymerisation of Coatings that Display Low Protein Binding Properties

### Part A: Europium Tagging of Human Serum Albumin (HSA)

Europium tagged human serum albumin (Eu-HSA) was prepared using the following method. HSA (Sigma, 99%, essentially fatty acid free) was labelled using a Delfia Europium labelling reagent (Perkin Elmer) overnight at 4° C. (pH 9.3). After separation of the Eu-labelled HSA from excess labelling reagent using Fast Protein Liquid Chromatography (FPLC) (Akta Purifier, GE Healthcare) on a Superdex 75 (30/10) size exclusion column (GE Healthcare), the Eu-HSA solution concentration was determined via amino acid analysis (Waters Alliance HPLC). The Eu:HSA labelling ratio was determined in the following manner. First a number of Eu standard solutions were prepared using various dilutions of a 100 nM Eu standard solution and Delfia Enhancement Solution (Perkin Elmer). The time resolved fluorescence counts from 100 μL aliquots of these solutions were then obtained using a PHERAStar multi-well plate reader (BMG Technologies, λₑₓ=337 nm, λₑₘ=620 nm, count delay 60 μs, count time 400 μs). Comparison of the time resolved fluorescence counts obtained from Eu-HSA solutions of known concentration and the Eu standards was used to calculate an Eu:HSA labelling ratio of 4.2.

### Part B: Plate Preparation

96 well tissue culture polystyrene plates (Nunclon™ Δ, Nunc) were coated with AAM and PEGMA-OMe graft polymers according to the experimental procedure described in Example 1. The wells were then thoroughly washed with with Milli-Q™ water using a plate washer (Thermo Wellwash 4 MK 2) and finally air dried. Subsequently, the plates were analysed for the amount of protein adsorption using an assay based on Eu-HSA.

### Part C: Protein Adsorption Assay

After UV graft polymerisation (Part B) each well was filled with 0.1 cm³ of a solution containing a solution of both Eu-HSA and HSA (1:1500 molar ratio) in phosphate buffered saline (PBS). The total HSA concentrations used were 100, 10 and 1 μg/cm³. The wells were incubated over 16 hours at room temperature, washed 6 times with PBS buffer solution and then treated with Delfia Enhancement Solution (Perkin Elmer) to release the Eu atoms from the adsorbed Eu-HSA. The solutions obtained in this way were then analysed via time resolved fluorescence using a PHERAStar multi-well plate reader (BMG Technologies, λₑₓ=337 nm, λₑₘ=620 nm, count delay 60 μs, count time 400 μs). The amount of adsorbed protein was quantified by comparison of the counts obtained from these solutions to a standard curve obtained with solutions of known Eu concentration.

The results from the protein adsorption assay, carried out using three different HSA concentrations, are shown in Table 2. At all protein concentrations, the adsorbed amount detected on commercially available 96 well tissue culture polystyrene plates [PS (Nunclon™ Δ)] far exceeded the amount detected on untreated polystyrene plates (PS, Nunc) and 96 well tissue culture polystyrene plates modified by UV graft polymerisation with AAM [PS (Nunclon™ Δ)-AAM] and PEGMA-OMe [PS (Nunclon™ Δ)-PEGMA-OMe]. The small amounts of protein detected on the AAM and in particular the PEGMA-OMe modified plates demonstrate that the UV graft polymerisation method can be used to produce low protein binding surface coatings.

TABLE 2
Quantification of the amount of protein adsorption onto surfaces before
and after UV graft polymerisation using Europium labelled HSA.

| | Protein concentration in solution | | |
|---|---|---|---|
| | (μg · cm⁻³) | | |
| | 100 | 10 | 1 |
| | Adsorbed amount (ng · cm⁻²) | | |
| PS (Nunclon ™ Δ) | 1101.0 | 153.5 | 18.0 |
| PS | 149.5 | 16.5 | 3.2 |
| PS (Nunclon ™ Δ) -AAM | 76.0 | 8.8 | 2.5 |
| PS (Nunclon ™ Δ)-PEGMA-OMe | 23.7 | 3.9 | 1.5 |

### Example 3

### UV Graft Polymerisation of Coatings with Low Cell Attachment Properties

### Part A: Plate Preparation:

A 24 well tissue culture polystyrene plate (Nunclon™ Δ, Nunc) was coated with UV graft polymers using AAM, PEGMA-OH and PEGMA-OMe monomer solutions as per the method described in Example 1. In the case of PEGMA-OH and PEGMA-OMe, the inhibitor was removed from the monomer solutions using a column filled with inhibitor removing beads (Sigma) before transfer of the solutions to a glove box. After UV grafting and subsequent rinsing with Milli-Q™ water as per Example 1, the plates were extracted in a large volume of Milli-Q™ water for at least 72 hours before drying in air.

### Part B: Cell Attachment Assay Using HeLa Cells

Surface modified 24 well plates were sterilised by the addition of sterile PBS (1 cm³/well, pH 7.4) which contained penicillin and streptomycin (Gibco) at concentrations of 120 and 200 μg·cm⁻³, respectively, for 4 hours at room temperature prior to cell seeding. HeLa cells were then seeded into each test well at a concentration of 2×10⁵ cells/well and incubated for 24 hours at 37° C. in humidified air containing 5% CO₂. The culture medium used was DMEM/Hams F12 (Gibco) containing 10% foetal bovine serum (FBS). After 20 hours incubation, four sample replicates were each treated with (3-(4,5-dimethylthiazol-2-yl)-2,5-diphenyltetrazolium bromide) (MTT) at 500 μg/cm³ in culture medium and incubated for a further 4 hours to obtain a quantitative value for cell attachment relative to a 24 well tissue culture polystyrene (Nunclon™ Δ, Nunc) control surface. Cell attachment on the control surface was set to 100% and attachment on all other surfaces was expressed as a percentage of the attachment on the control surface. The MTT containing solution was removed from wells after the 4 hour incubation step. Wells were then washed three times with sterile PBS prior to the release of the MTT formazan crystals from the cells using 1 cm³/well of DMSO. The plates were placed on a plate shaker and gently agitated for 15 minutes to ensure crystals were dissolved and solutions well mixed. 100 μl samples from each well were then transferred into wells of a 96 well plate for optical density measurements at λ=595 nm test wavelength and λ=655 nm reference wavelength. At 20 hours, immediately prior to the addition of the MTT, the cells were also viewed by phase contrast microscopy (Olympus IMT-2, 10× objective lens) and representative images of cell attachment were recorded digitally.

Presented in Table 3 are the results obtained from the MTT assay. The results clearly show that HeLa cell attachment was reduced to very low levels on surfaces modified with AAM, PEGMA-OH and PEGMA-OMe UV graft polymers in comparison to cell attachment on the PS (Nunclon™ Δ) control surface. This result was further supported by phase contrast images shown in FIG. 2A and FIG. 2B. Here, the appearance of HeLa cells observed after 20 hours on AAM, PEGMA-OH and PEGMA-OMe UV graft polymer coatings was similar, showing aggregated cells that had not attached on the surface or loosely attached, rounded cells that were easily removed from the surface by shaking or rinsing. As an example, presented in FIG. 2A is a representative image of HeLa cells on an AAM modified surface. In comparison, HeLa cells appeared firmly adherent and well spread after this culture period on a PS (Nunclon™ Δ) control surface (FIG. 2B).

TABLE 3
HeLa cell attachment, obtained via MTT assay, after 24 hours culture on
various surfaces. Cell attachment on the polymer grafted surfaces was
normalised to that obtained on the PS (Nunclon ™ Δ) control surface.

| Sample | % cell attachment | sd |
|---|---|---|
| PS (Nunclon ™ Δ)-AAM | 2.30 | 0.30 |
| PS (Nunclon ™ Δ)-PEGMA-OH | 0.82 | 0.71 |
| PS (Nunclon ™ Δ)-PEGMA-OMe | 0.52 | 0.08 |
| PS (Nunclon ™ Δ) | 100.00 | 3.28 |

### Example 4

### UV Graft Polymerisation of Coatings from Acrylic Acid (AA) and Acrylamide (AAM) and Subsequent Activation and Covalent Binding of Amines

Part A: UV Grafting of Coatings from AA and AAM

As described in Example 1, 5% (w/v) aqueous solutions of acrylic acid (AA) and acrylamide (AAM) were degassed by purging with nitrogen for more than 15 min in a glove box. The solutions were then mixed to yield AAM monomer solutions containing 5%, 10%, 20% and 50% (v/v) AA monomer. Solutions made in this way were then transferred into the wells of 96 well plates (0.15 cm³ per well).

While still in the glove box, plates containing the solutions described above were then vacuum sealed into polymer bags (Sunbeam FoodSaver) and removed from the glove box. The plates were then passed under a UV lamp (FUSION Systems) 30 times on a conveyor belt at a speed of approximately 1.8 m/min as described in Example 1. After each pass the plate was turned 90 degrees to enable more uniform UV irradiation. The wells of the plates were then thoroughly washed with Milli-Q™ water using a plate washer (Thermo Wellwash 4 MK 2) and air dried. For XPS analysis, the bottom of the wells of interest was removed using a cutting tool.

Analysis of the data presented in FIG. 3 and Table 4 clearly showed that UV graft polymerisation of the monomer mixtures resulted in successful coatings. Furthermore, these results demonstrate that the composition of the UV graft polymer coatings can be controlled by choosing an appropriate monomer mixture. XPS spectra of the coatings prepared with an increasing AA ratio in the monomer feed (see FIG. 3) contained an increased contribution from the C5 component, which originated from the carboxylic acid functionality (from the AA monomer) in the coatings. At a monomer composition of 5% AA, the C4 signal originating from amide bonds (from the AAM monomer) was dominant while at a monomer composition of 50% AA, the C4 and C5 signals were almost equivalent in intensity.

TABLE 4
The components obtained by curve fitting the XPS C 1s high resolution
spectra presented in FIG. 3. The monomer composition is expressed as the
percentage of AA in the monomer mixture, the remaining comprising
the AAM monomer.

| Monomer composition | C1 + C2 | C3 | C4 | C5 |
|---|---|---|---|---|
| 5% AA | 77.7 | 0.8 | 20.8 | 0.6 |
| 10% AA | 73.0 | 0.2 | 25.2 | 1.6 |
| 20% AA | 71.1 | 1.0 | 17.7 | 7.0 |
| 50% AA | 73.5 | 0.42 | 11.6 | 14.5 |

The elemental ratios presented in Table 5 confirm these results. With increasing AA content in the monomer feed, the amount of oxygen present in the copolymer coating increased, while the nitrogen content decreased.

TABLE 5
Elemental ratios calculated from XPS survey spectra obtained from
UV graft polymer coatings prepared from mixtures of AA and AAM
monomer solutions. The monomer composition is expressed by the
percentage of AA in the monomer mixture.

| Monomer composition | O/C | N/C |
|---|---|---|
| 5% AA | 0.288 | 0.228 |
| 10% AA | 0.317 | 0.233 |
| 20% AA | 0.329 | 0.182 |
| 50% AA | 0.425 | 0.109 |

### Part B: NHS Activation of Carboxylic Acid Surface Functional Groups

A solution containing 0.125 M 1-ethyl-3-(3-dimethylaminopropyl) carbodiimide (EDC) and 0.125 M N-hydroxysuccinimide (NHS) in Milli-Q™ water was prepared and 0.05 cm³ placed into each well of a plate prepared as described in Part A. The solution was added to the wells immediately after preparation. After 20 minutes incubation the wells were washed 3 times with Milli-Q™ water in a plate washer (Thermo Wellwash 4 MK 2) and dried using a stream of nitrogen. The NHS activated plate was then immediately used for subsequent reactions.

### Part C: Covalent Immobilisation of Amines on NHS Activated Graft Polymer Coatings

A 0.1 M TFEA solution was prepared using Milli-Q™ water, 0.05 cm³ aliquots of which were then transferred into each well of a plate freshly prepared as described in Part B. After incubation for 24 hours, the wells were then thoroughly washed with Milli-Q™ water, air dried and analysed by XPS.

The increase in the F/C ratio obtained with increasing AA content (Table 6) demonstrated that the activation of the acrylic acid with NHS was successful and that NHS activated surfaces prepared in this way were highly reactive towards amine containing molecules.

TABLE 6
Elemental ratios calculated from XPS survey spectra obtained on UV
graft polymer coatings made from mixtures of AA and AAM
monomer solutions after subsequent NHS activation and reaction
with TFEA. The monomer composition is expressed by the
percentage of AA in the monomer mixture.

| Monomer composition | O/C | N/C | F/C |
|---|---|---|---|
| 5% AA | 0.286 | 0.249 | 0.008 |
| 10% AA | 0.260 | 0.221 | 0.035 |
| 20% AA | 0.285 | 0.216 | 0.058 |
| 50% AA | 0.366 | 0.175 | 0.243 |

### Example 5

### UV Graft Polymerisation of Coatings from PEGMA-OH and Subsequent Activation and Covalent Binding of Amines

Si-ALAPP samples with a size of 1 cm×1 cm were prepared as described in Example 1. As per Example 1, a 5% (w/v) solution of PEGMA-OH was prepared in Milli-Q™ and degassed by purging with nitrogen for 30 min in a glove box. In a glove box, to each well of a 24 well tissue culture polystyrene plate (Nunclon™ Δ, Nunc), was added a Si-ALAPP wafer as well as 0.6 cm³ of the PEGMA-OH solution.

Whilst still in the glove box, the 24 well plate containing Si-ALAPP samples and PEGMA-OH monomer solution was vacuum sealed into polymer bags (Sunbeam FoodSaver) and removed from the glove box. The plates were passed 20 times under a UV lamp (FUSION Systems) on a conveyor belt at a speed of approximately 1.8 m/min as described in Example 1. After each pass the plate was rotated 90 degrees to enable more uniform UV irradiation. Subsequently the samples were removed from the plate, thoroughly washed with Milli-Q™ water and air dried. The samples were then immersed for 2 hours into a 0.5 M solution of carbonyl diimidazole (CDI) in dry DMSO. The samples were then rinsed with Milli-Q™ water and subsequently immersed into a 0.1 M solution of TFEA in PBS buffer (pH 7.4) for 7 days. Finally the samples were washed with Milli-Q™ water, air dried and analysed by XPS.

Complete attenuation of the nitrogen signal from the ALAPP coating, as may be observed from the data presented in Table 7, after UV graft polymerisation of PEGMA-OH indicated successful coating with a thickness of greater than 10 nm (XPS sampling depth). After reaction of the OH groups of the PEGMA-OH groups with CDI, an increase in the N/C ratio was observed (Table 7), demonstrating a successful surface activation reaction. Finally the presence of fluorine after the reaction of TFEA with the CDI activated surface (Table 7) demonstrated the reactivity of surfaces prepared in this way towards amine containing molecules.

TABLE 7
Elemental ratios calculated from XPS survey spectra obtained
on PEGMA-OH UV graft polymer coatings before and after
subsequent CDI activation and reaction with TFEA.

| Sample | O/C | N/C | F/C |
|---|---|---|---|
| Si-ALAPP-PEGMA-OH | 0.468 | 0.000 | 0.000 |
| Si-ALAPP-PEGMA-OH-CDI | 0.453 | 0.032 | 0.000 |
| Si-ALAPP-PEGMA-OH-CDI-TFEA | 0.458 | 0.018 | 0.018 |

### Example 6

### UV Graft Polymerisation of Coatings from NIPAM for Thermo-Responsive Coatings

Part A: UV Grafting of Coatings from NIPAM

Tissue culture polystyrene plates (4 well, Nunclon™ Δ, Nunc) were used as received. UV graft polymer coatings on these substrates were carried out using N-isopropylacrylamide (NIPAM) monomer as per Example 1. In a glove box under a nitrogen atmosphere, a 5% (w/v) solution of NIPAM was prepared in Milli-Q™ water and purged for 30 min with nitrogen. Aliquots of this monomer solution (0.6 cm³) were then transferred into each well of the 4 well plates. Whilst still in the glove box, the plates were vacuum sealed into polymer bags (Sunbeam FoodSaver) and removed from the glove box. The plates were passed 20 times under a UV lamp (FUSION Systems) on a conveyor belt at a speed of approximately 1.8 m/min as per Example 1. After each pass the plate was rotated 90 degrees to enable more uniform UV irradiation. Subsequently the plates were washed five times with Milli-Q™ water followed by immersion of the plates in a large volume of Milli-Q™ water over 72 hours. Finally the surface modified plates were air dried.

### Part B: L929 Cell Attachment on NIPAM Graft Polymer Modified Cell Culture Substrates

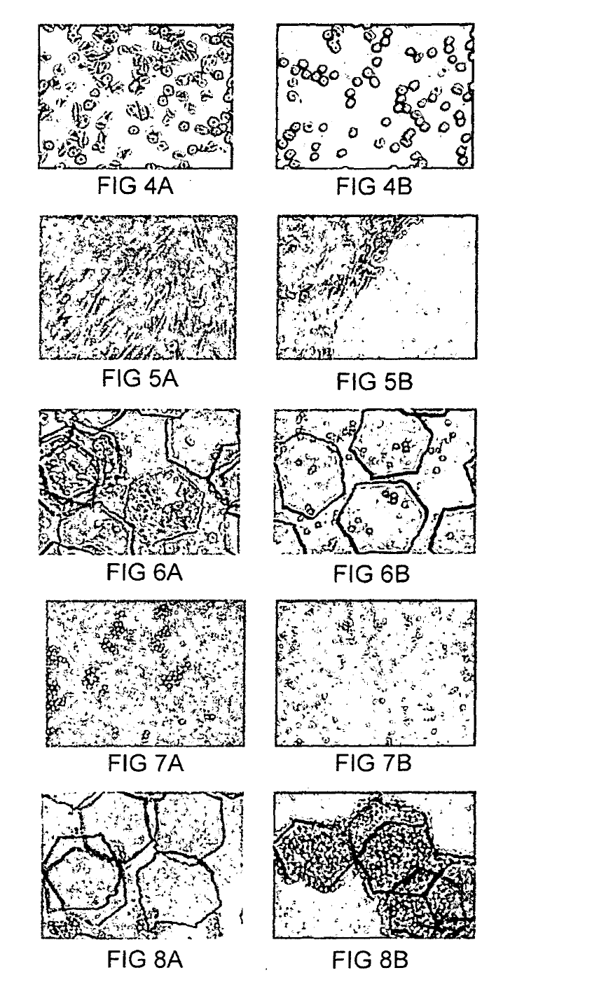

Mouse fibroblast cells (L929) were cultured in modified Eagles medium (MEM) containing 10% foetal bovine serum (FBS) and 1% non-essential amino acids. The wells of the plates were sterilised by the addition of sterile PBS (0.8 cm³/well, pH 7.4) which contained 2% (v/v) of an antibiotic-antimycotic solution (anti-anti, Gibco), respectively, for 2-4 hours at room temperature prior to cell seeding. L929 cells were seeded onto NIPAM UV graft polymer modified 4 well plates (described in Part A) at a seeding density of 2×10⁵ cells/well. After an incubation period of 24 hours, a phase contrast image of cell attachment was taken of a representative sample while maintaining the plate at a temperature of 37° C. on a heated microscope stage. Subsequently the heated stage was removed and the plate was allowed to cool down to 20° C. 30 min after removing the heated stage another image was recorded. The phase contrast images taken at 37° C. and 20° C. respectively are shown in FIG. 4A and FIG. 4B. The cells in the image taken at 37° C. (FIG. 4A) were adherent and of well spread morphology whilst the cells in the image taken at 20° C. (FIG. 4B) were of rounded morphology and could be easily washed off the surface. These cell culture results demonstrate the thermo-responsive nature of the NIPAM UV graft polymer coating, which leads to cell-adhesive properties at physiological temperature (37° C.) and non-cell adhesive properties at room temperature (20° C.).

### Part C: MSC Attachment on NIPAM Graft Polymer Modified Cell Culture Substrates

Mesenchymal stem cells (MSCs) were isolated from human bone marrow aspirates using standard methodologies. That is, bone marrow was first separated over a density gradient (1.077 gms/100 cm³) and the light density fraction collected, washed in PBS and then cells resuspended in α-MEM media supplemented with 20% FBS (pre-selected batches). Cells were then added to T-flasks at a density of approximately 5×10⁵ cells/cm³ and incubated at 37° C. for 2-3 days. After this, the non-adherent fraction was removed followed by gentle washing with fresh media and then PBS, leaving behind adherent MSCs. Fresh alpha-MEM and FBS was added and the cells cultured for 7 days. They were then split (˜1:3, depending on the confluency) by using EDTA and Ca and Mg free PBS and replated with fresh media and serum.

Human MSCs were transferred into a-MEM media supplemented with 20% FBS and 5 ng/cm³ human recombinant FGF-2 (Prospec) and seeded onto NIPAM UV graft polymer modified 4 well plates (described in Part A) at a seeding density of 1×10⁵ cells/well. Prior to cell seeding, the wells of the plates were sterilised by the addition of sterile PBS (0.3 cm³/well, pH 7.4) which contained 2% (v/v) of an antibiotic-antimycotic solution (Gibco), respectively, for 2-4 hours at room temperature. After an incubation period of 24 hours in a humidified incubator at 37° C. and 5% CO₂, a phase contrast image of cell attachment was taken on of representative region of the well while maintaining the plate at a temperature of 37° C. on a temperature controlled microscope stage. Subsequently the temperature controlled stage was removed and the plate was allowed to cool down to 20° C. 30 min after removing the heated stage another image was recorded. The phase contrast images taken at 37° C. and 20° C. respectively are shown in FIG. 5A and FIG. 5B. The cells in the image taken at 37° C. (FIG. 5A) were adherent, well spread and almost at confluence whilst in the image taken at 20° C. (FIG. 5B) the cells were aggregated and had lifted off the surface in some regions. These human MSC culture results again demonstrate the thermo-responsive nature of the NIPAM UV graft polymer coating, which leads to cell-adhesive properties at physiological temperature (37° C.) and non-cell adhesive properties at room temperature (20° C.).

### Example 7

### UV Graft Polymerisation of Coatings from NIPAM for Thermo-Responsive Coatings on Microparticles

Part A: UV Grafting of Coatings from NIPAM on Microcarrier Particles

Tissue culture polystyrene plates (24 well, Nunclon™ Δ, Nunc) were used as received. 50 mg of MicroHex™ microcarrier particles (Nunclon™ Δ, Nunc) were added to each well of these plates. UV graft polymer coatings on the microcarrier particles were prepared out using N-isopropylacrylamide (NIPAM) monomer as per Example 1. Briefly, in a glove box under a nitrogen atmosphere, a 5% (w/v) solution of NIPAM was prepared in Milli-Q™ water and purged for 30 min with nitrogen. Aliquots of this monomer solution (0.6 cm³) were transferred into each well of the 24 well plates containing the microparticles. Whilst still in the glove box, the plates were then vacuum sealed into polymer bags (Sunbeam FoodSaver) and removed from the glove box. The plates were passed 20 times under a UV lamp (FUSION Systems) on a conveyor belt at a speed of approximately 1.8 m/min as per Example 1. After each pass the plate was gently agitated and rotated 90 degrees to enable more uniform UV irradiation. Subsequently the microparticles were washed ten times with Milli-Q™ water, centrifuging and resuspending the microparticles after each washing step. Finally the surface modified microparticles were incubated in a large volume of Milli-Q™ water over 72 hours before drying under vacuum.

The MicroHex™ microcarrier particles were analysed by XPS before and NIPAM UV graft polymerisation. Analysis of the results presented in Table 8 demonstrated that the surface coating procedure was successful. In particular the increase in the calculated N/C ratio indicated the presence of the graft polymer layer on the surface of the microcarrier particles.

TABLE 8
Elemental ratios calculated from XPS survey spectra obtained
on MicroHex ™ microcarrier particles (Nunclon ™ Δ, Nunc)
before and after coating with a NIPAM UV graft polymer.

| | O/C | N/C |
|---|---|---|
| MicroHex ™ | 0.166 | 0.001 |
| MicroHex ™-NIPAM | 0.157 | 0.123 |

### Part B: L929 Cell Attachment on NIPAM Graft Polymer Modified Microcarrier Particles

Mouse fibroblast cells (L929) were cultured in modified Eagles medium (MEM) containing 10% foetal bovine serum and 1% non-essential amino acids. L929 cells were seeded onto NIPAM UV graft polymer modified microparticles (described in Part A) contained in the wells of a 4 well tissue culture polystyrene plate (Nunclon™ Δ, Nunc) that had been modified with a non-cell adhesive PEGMA-OH UV graft polymer coating as per Example 3. Each well contained 0.1 cm³ of packed surface modified particles. Prior to cell seeding the surface modified microcarrrier particles were sterilised by the addition of sterile PBS (0.6 cm³/well, pH 7.4) which contained 2% (v/v) of an antibiotic-antimycotic solution (Gibco), respectively, for 2-4 hours at room temperature. The cell seeding density was 2×10⁴ cells/well. After a cell culture period of 24 hours, a phase contrast image of cell attachment was taken of a representative sample while maintaining the plate at a temperature of 37° C. on a heated microscope stage. Subsequently the heated stage was removed and the plate was allowed to cool down to 20° C. 30 min after removing the heated stage another image was recorded. The phase contrast images taken at 37° C. and 20° C. respectively are shown in FIG. 6A and FIG. 6B. The cells in the image taken at 37° C. (FIG. 6A) were adherent and of partially well spread morphology whilst the cells in the image taken at 20° C. (FIG. 6B) were of rounded morphology which could easily be washed off the surface. These cell culture results demonstrate the effectiveness of the thermo-responsive NIPAM UV graft polymer coating on microparticles, which leads to cell-adhesive properties at physiological temperature (37° C.) and non-cell adhesive properties at room temperature (20° C.).

### Example 8

### Evenness of Coatings on Three-Dimensional Substrates

Tissue culture polystyrene plates (96 well, Nunclon™ Δ, Nunc) were used as received. UV graft polymer coatings on these plates were obtained using AAM monomer as per Example 1. Briefly, in a glove box (under a nitrogen atmosphere containing <0.2% oxygen), 250 mg of AAM were dissolved in 5 cm³ Milli-Q™ water and the solution purged for 10 min with nitrogen to remove residual oxygen. Each well of the 96 well plates was then filled with 0.15 cm³ of the monomer solution. After vacuum sealing of the plates into polymer bags (Sunbeam FoodSaver) in the glove box, the plates were placed onto a conveyor belt which ran underneath a UV lamp (λ˜200-450 nm, maximum intensity 360-390 nm, length of lamp 15 cm, output 1.8 kW, FUSION systems). The average belt speed was kept at 1.8 m·min⁻¹ to give an irradiation time of approximately 4 sec per pass. The vacuum sealed plates were exposed to UV irradiation during 30 passes. The orientation of the plates was rotated by 90 degrees after each pass. The wells were then thoroughly washed with Milli-Q™ water using a plate washer (Thermo Wellwash 4 MK 2) and finally air dried. For XPS analysis, the bottom as well as the wall of selected wells on the 96 well plate was removed using a cutting tool.

Presented in Table 9 are the elemental ratios, obtained by XPS analysis, after UV graft polymerisation on different regions of a representative well. Due to the fact that the well was filled with 0.15 cm³ of the monomer solution during UV graft polymerisation, a coating was expected on all regions of the well except the top of the wall. This expectation was confirmed by the results shown in Table 9. The O/C and N/C ratios obtained on top of the wall (approximately 2 mm from the top) were similar to those obtained on other 96 well tissue culture polystyrene samples (Nunclon™ Δ, Nunc) (see Table 1), suggesting that this part of the plate was not affected by the UV graft polymerisation process. However, the significant changes in the O/C and N/C ratio observed for each of the other regions in comparison to the unmodified top of the wall suggest a successful AAM graft polymer coating in each case. In addition, the elemental ratios obtained from the bottom of the well, the lower part of the wall (approximately 2 mm from the bottom) and the middle part of the well (half way up the wall) show similar O/C and N/C values close to the theoretically expected values, suggesting an even coating thickness of more than 10 nm (XPS sampling depth) throughout the surface modified region of the well.

TABLE 9
Elemental ratios calculated from XPS survey spectra obtained
on a single well of a partially AAM UV graft polymer
modified 96 well plate. Spectra were recorded on the different
regions present in the well. These are the bottom of the
well, the lower part of the wall (approximately 2 mm from the
bottom), the middle part of the well (half way up the wall) and
the top part of the wall (approximately 2 mm from the top).

| Region | O/C | N/C |
|---|---|---|
| Bottom of well | 0.259 | 0.259 |
| Wall (bottom) | 0.259 | 0.248 |
| Wall (middle) | 0.259 | 0.255 |
| Wall (top) | 0.085 | 0.010 |

### Example 9

### UV Graft Copolymerisation of Coatings from AA and AAM on 96 Well Substrates, Covalent Immobilisation of a Peptide and Effect on the Cellular Response

Part A: UV Grafting of Copolymer Coatings from AA and AAM

As per Example 1, 5% (w/v) aqueous solutions of acrylic acid (AA) and acrylamide (AAM) were degassed by purging with nitrogen for more than 15 min in a glove box. A solution was made from these by mixing 10% (v/v) AA and 90% (v/v) AAM monomer solution (10% AA). The solution prepared in this way was then transferred into the wells of 96 well plates (0.15 cm³ per well).

While still in the glove box, plates containing the solutions described above were then vacuum sealed into polymer bags (Sunbeam FoodSaver) and removed from the glove box. The plates were then passed under a UV lamp (FUSION Systems) 30 times on a conveyor belt at a speed of approximately 1.8 m/min as described in Example 1. After each pass the plate was rotated 90 degrees to enable more uniform UV irradiation. The wells of the plates were then thoroughly washed with Milli-Q™ water using a plate washer (Thermo Wellwash 4 MK 2) and air dried.

Part B: NHS Activation of Carboxylic Acid Surface Functional Groups and Covalent Immobilisation of cRGD

A solution containing 0.125 M 1-ethyl-3-(3-dimethylaminopropyl) carbodiimide (EDC) and 0.125 M N-hydroxysuccinimide (NHS) in Milli-Q™ water was prepared and 0.05 cm³ placed into each well of a 10% AA modified 96 well plate prepared as described in Part A. The solution was added to the wells immediately after preparation. After 20 minutes incubation the wells were washed 3 times with Milli-Q™ water in a plate washer (Thermo Wellwash 4 MK 2). The NHS activated plate was then immediately used for subsequent reactions.

An N—C terminally cyclised molecule containing a tri-amino acid motif, arginine-glycine-aspartic acid (c(RGDfK), Peptides International) was covalently attached to the polymer coating using the following method. Aliquots of a solution (0.1 cm³) containing 200 μg/mL of c(RGDfK) in PBS were added to each well of a freshly prepared NHS activated plate described above. The solution was incubated in the wells overnight (15 h) at 4° C., after which the solution was removed and the wells washed 10 times with PBS.

### Part C: L929 Cell Attachment on Surface Modified 96 Well Plates

Mouse fibroblast cells (L929) were cultured in modified Eagles medium (MEM) containing 10% foetal bovine serum (FBS) and 1% non-essential amino acids. The wells of the plates were sterilised by the addition of sterile PBS (0.3 cm³/well, pH 7.4) which contained 2% (v/v) of an antibiotic-antimycotic solution (Gibco), respectively, for 2-4 hours at room temperature prior to cell seeding. L929 cells were seeded onto the wells of the plate with covalently immobilised c(RGDfK), freshly prepared as described in Part B, at a seeding density of 2×10⁴ cells/well. In addition, cells were also seeded into the wells of a freshly prepared plate which had not been NHS activated and onto which c(RGDfK) had not been covalently immobilised. After an incubation period of 20-22 hours, phase contrast images of cell attachment were taken of representative regions. The phase contrast images taken are shown in FIG. 7A and FIG. 7B. The cells in the image for the sample which did not contain covalently immobilised c(RGDfK) (FIG. 7A) were non-adherent and of rounded morphology whilst the cells in the image taken for the sample to which c(RGDfK) had been covalently immobilised (FIG. 7B) were adherent and of a well spread morphology. These cell culture results demonstrate the cell adherent properties of the UV graft polymer coating formed from a monomer feed of 10% AA and 90% AAM, to which c(RGDfK) had been covalently immobilised and the non-cell adherent nature of the UV graft coating to which c(RGDfK) had not been covalently immobilised.

### Example 10

### UV Graft Copolymerisation of Coatings from AA and AAM on MicroHex™ Substrates, Covalent Immobilisation of a Peptide and Effect on the Cellular Response

Part A: UV Grafting of Copolymer Coatings from AA and AAM on Microcarrier Particles

Tissue culture polystyrene plates (24 well, Nunclon™ Δ, Nunc) were used as received. 50 mg of MicroHex™ microcarrier particles (Nunclon™ Δ, Nunc) were added to each well of these plates. UV graft polymer coatings on the microcarrier particles were prepared as per Example 7. Briefly, in a glove box under a nitrogen atmosphere, 5% (w/v) aqueous solutions of acrylic acid (AA) and acrylamide (AAM) were degassed by purging with nitrogen for more than 15 min in a glove box. A solution was made from these by mixing 10% (v/v) AA and 90% (v/v) AAM monomer solutions (10% AA). Aliquots of this 10% AA monomer mixture (0.6 cm³) were transferred into each well of the 24 well plates containing the microparticles. Whilst still in the glove box, the plates were then vacuum sealed into polymer bags (Sunbeam FoodSaver) and removed from the glove box. The plates were passed 30 times under a UV lamp (FUSION Systems) on a conveyor belt at a speed of approximately 1.8 m/min as per Example 1. After each pass the plates were gently agitated and rotated 90 degrees to enable more uniform UV irradiation. Subsequently the microparticles were washed ten times with Milli-Q™ water, centrifuging and resuspending the microparticles after each washing step. Finally the surface modified microparticles were incubated in a large volume of Milli-Q™ water over 72 hours before drying under vacuum prior to XPS analysis.

The results of XPS analysis presented in Table 10 demonstrate clearly that the graft polymerisation was successful on the microcarrier particles. Both the O/C and N/C atomic ratios were observed to increase after the graft polymerisation reaction. A comparison of the relative O/C and N/C ratio indicated a coating which contained both the AAM and AA monomers. This was confirmed by analysis of the high resolution C 1s spectra (not shown).

TABLE 10
Elemental ratios calculated from XPS survey spectra obtained on
MicroHex ™ microcarrier particles (Nunclon ™ Δ, Nunc) before
and after coating with a UV graft polymer made from mixtures
of 10% (v/v) AA and 90% (v/v) AAM monomer solutions.

| | O/C | N/C |
|---|---|---|
| MicroHex ™ | 0.166 | 0.001 |
| MicroHex ™-10% AA | 0.258 | 0.149 |

Part B: NHS Activation of Carboxylic Acid Surface Functional Groups and Covalent Immobilisation of c(RGDfK)

A solution containing 0.125 M 1-ethyl-3-(3-dimethylaminopropyl) carbodiimide (EDC) and 0.125 M N-hydroxysuccinimide (NHS) in Milli-Q™ water was prepared and incubated with 10% AA modified MicroHex™ microcarrier particles in such a way that the particles were covered by an excess of the solution. The solution was added to the modified microparticles immediately after preparation. After 20 minutes incubation with occasional shaking, the microparticles were washed 3 times with Milli-Q™ water by centrifugation and resuspension in Milli-Q™ water. The NHS activated microparticles were then immediately used for subsequent reactions.

An N—C terminally cyclised molecule containing a tri-amino acid motif, arginine-glycine-aspartic acid (c(RGDfK), Peptides International) was covalently attached to the polymer coating using the following method. Aliquots of a solution (0.6 cm³) containing 200 μg/mL of c(RGDfK) in PBS were added to each well containing freshly prepared NHS activated MicroHex™ microcarrier particles prepared as described above. Subsequently the microparticles were washed ten times with Milli-Q™ water, centrifuging and resuspending the microparticles after each washing step. Finally the surface modified microparticles were incubated in a large volume of Milli-Q™ water over 72 hours prior to cell culture experiments.

### Part C: L929 Cell Attachment on Surface Modified Microcarrier Particles

Mouse fibroblast cells (L929) were cultured in modified Eagles medium (MEM) containing 10% foetal bovine serum and 1% non-essential amino acids. L929 cells were seeded onto 10% AA modified microparticles (described in Part A) and c(RGDfK) modified microparticles (described in Part B), respectively, contained in the wells of a 4 well tissue culture polystyrene plate (Nunclon™ Δ, Nunc) that had been modified with a non-cell adhesive PEGMA-OH UV graft polymer coating as per Example 3. Prior to cell seeding the micocarrier particles were sterilised by the addition of sterile PBS (0.3 cm³/well, pH 7.4) which contained 1% (v/v) of an antibiotic-antimycotic solution (Gibco), respectively, for 2-4 hours at room temperature. Each well contained 0.1 cm³ of packed surface modified particles. The cell seeding density was 2×10⁴ cells/well. After a cell culture period of 20-22 hours, a phase contrast image of cell attachment was taken of a representative region for each of the samples. The phase contrast images taken are shown in FIG. 8A and FIG. 8B. The cells in the image for the sample which did not contain covalently immobilised c(RGDfK) (FIG. 8A) were non-adherent and of rounded morphology whilst the cells in the image taken for the sample to which c(RGDfK) had been covalently immobilised (FIG. 8B) were adherent and of a well spread morphology. These cell culture results demonstrate the cell adherent properties of the UV graft polymer coating formed from a monomer feed of 10% AA and 90% AAM, to which c(RGDfK) had been covalently immobilised and the non-cell adherent nature of the UV graft coating to which c(RGDfK) had not been covalently immobilised thermo-responsive thermo-responsive thermo-responsive.

### Example 11

### Polymer Grafting: Continuous Versus Intermittent UV Irradiation

### Part A: Preparation of Coatings

Silicon wafers (Si) were cut into squares of approximately 7×7 mm dimension, ultrasonically cleaned in a 2% (v/v) RBS-35® surfactant solution, rinsed with ethanol, thoroughly rinsed with Milli-Q™ water, and dried under purified nitrogen. Si wafer pieces were further cleaned by UV/ozone treatment in a ProCleaner™ instrument (Bioforce Nanoscience, USA) for 60 minutes immediately before use. Onto the Si wafer pieces was deposited a cross-linked, polymeric thin film with amine functionality from allylamine monomer using radio-frequency glow discharge (RFGD) techniques. The reaction chamber was completely evacuated to a pressure of <0.003 mbar and then filled with allylamine vapour to a slowly rising pressure of 0.200 mbar. At this time, voltage was applied across the electrodes at a frequency of 200 kHz and load power of 20 W for a period of 25 seconds. The resultant allylamine coatings (Si-ALAPP) were then rinsed in Milli-Q™ water before further use.

Solutions of AAM were prepared in H₂O at a concentration of 10% (w/v) in a N₂ glove box and purged, with N₂, for 60 minutes. AAM solution was then applied to Si-ALAPP samples so that the AAM solution was 3 mm deep and sealed against oxygen ingress inside a polypropylene bag using a domestic vacuum food storage system (Sunbeam). The sealed samples were then removed from the N₂ glove box and exposed to UV radiation generated by a high powered UV lamp (Fusion Systems FS300s with 9 mm D-bulb). In normal operation, the sample is passed under the lamp on a conveyor (Fusion UV Systems, Inc. LC6B Benchtop Conveyor) with each pass resulting in 2849 (UVA), 822 (UVB), 81.5 (UVC), and 2922 (UVV) mJ/cm² (as measured with a portable UV meter (EIT UV Power Puck II)). Knowing these values it was possible to equalise the UV radiation dose across different processing protocols. The protocols tested were 1) intermittent (22 passes on belt), 2) intermittent+delay (22 passes with a 30 second delay between each), and 3) continuous (the sample fixed in place under the lamp for a time that delivered the equivalent UV radiation dose as 22 passes). After the desired UV radiation exposure, the samples were washed copiously with water and then dried under a filtered purified nitrogen stream prior to analysis.

### Part B: Characterisation of Coatings

XPS analysis of the coatings prepared was carried out and the results obtained are presented in Table 11. After RFGD thin film deposition (Si-ALAPP) the composition of the sample was as expected for coatings of this sort, being rich in C and N. After continuous irradiation of the sample in AAM monomer solution, both the O and N atomic percentages increased, consistent with a graft polymer coating comprising mainly polyacrylamide (compare to PAAM theoretical composition). Irradiation of the samples in the presence of AAM monomer solution in an intermittent fashion and an intermittent+delay fashion also produced coatings which conformed to the theoretical expectations for a graft polyacrylamide coating. High resolution C 1s spectra (not shown) confirmed the presence of polyacrylamide coatings in all three cases. It is generally not possible to estimate the thickness of coatings with the XPS technique unless the thickness is less than the analysis depth of the XPS technique (5-10 nm).

TABLE 11
Atomic percentages and elemental ratios obtained from XPS
analysis of graft polymer coatings formed using continuous,
intermittent or intermittent + delay conditions. Also included
for comparison are the analysis results obtained for the Si-ALAPP
sample and the theoretical composition of polyacrylamide (PAAM).

| | C | O | N | | |
|---|---|---|---|---|---|
| Sample | (at. %) | (at. %) | (at. %) | O/C | N/C |
| PAAM (theoretical) | 60.7 | 19.7 | 19.7 | 0.33 | 0.33 |
| Si-ALAPP | 76.8 | 12.6 | 10.4 | 0.16 | 0.14 |
| Continuous | 64.6 | 18.3 | 17.1 | 0.28 | 0.27 |
| Intermittent | 62.3 | 18.8 | 16.8 | 0.30 | 0.27 |
| Intermittent + delay | 60.4 | 19.6 | 16.9 | 0.32 | 0.28 |

In order to estimate the thickness of the graft polymer coatings formed using the three conditions under investigation, profilometry was used. In this case, coated samples were scratched with the tip of a syringe needle to expose the Si wafer beneath. The depth of the scratch was then measured with a profilometer (Dektak by Veeco) for various scratches/locations and referenced to the Si-ALAPP substrate surface. The results of replicate profilometry experiments are presented in Table 12. The data for the thickness of the Si-ALAPP sample is not included (typically 25-30 nm thick). Thickness data for graft polymer coatings are referenced to the surface of the Si-ALAPP substrate.

Clearly indicated by the data presented in Table 12 was that the dry thickness of the coatings prepared increased in the order: continuous<intermittent<intermittent+delay. Clearly the dry thickness coating produced using continuous UV irradiation was significantly lower than that of the two coatings produced under intermittent UV irradiation conditions. Furthermore, the coating made using intermittent+delay UV irradiation was significantly thicker than the coating made with intermittent UV irradiation.

For cell culture applications, the hydrated thickness of the coatings produced is of more relevance than the dry thickness. In order to estimate the hydrated thickness of the coatings, an Atomic Force Microscope (AFM) technique was implemented. Here a silica colloid particle (diameter ˜4 μm) was glued (Epon 1004, Shell) to the cantilever spring to provide a probe of known geometry (i.e. spherical). Interactions between the silica colloid and the graft polymeric coatings in phosphate buffered saline (PBS) (pH 7.4) solution were then measured as a function of the separation distance. As the silica colloid makes contact with the coating, a repulsive force is generated, the range of which provides an estimate of the hydrated thickness of the coating. Interaction forces were measured using an MFP-3D AFM (Asylum Research, Santa Barbara, Calif.) at several locations on the samples which were mounted inside a fluid cell. The spring constant of the cantilever was determined using the resonance method of Cleveland et al. (Cleveland, J. et al., (1993), Rev. Sci. Instrum., 64, 403-5) and the radius of the silica particle was determined using optical microscopy. The deflection of the cantilever as a function of piezo distance travelled was scaled to the force as a function of separation distance using the MFP-3D software. Reference measurements were made using a non-compressible surface such as a Si wafer piece and the inverse slope of the deflection in hard contact was used to calibrate the photodetector.

TABLE 12
Results obtained for graft polymeric coatings prepared by continuous,
intermittent or intermittent + delay conditions. Data were obtained for
the dry thickness using profilometry. The data for the hydrated thickness
was obtained using AFM direct force measurements in PBS solution.
The swelling ratio was calculated from the dry and hydrated thickness
values. The modulus of the samples was obtained from analysis of the
interaction force data and comparison to theoretical predictions calculated
using Hertz theory (reference for Hertz theory and the approach used is:
Dimitriadis, E.K., et al. (2002), Biophysical J., 82, 2798-2810). Reported
results are averages obtained from analysis of three locations on replicate
samples.

| | Dry | Hydrated | | |
|---|---|---|---|---|
| | Thickness | Thickness | Swelling | Modulus |
| Sample | (nm) | (nm) | Ratio | (Pa) |
| Continuous | 136 ± 38 | 708 ± 20 | 5.2 | 200 |
| Intermittent | 231 ± 11 | 4056 ± 155 | 17.5 | 90 |
| Intermittent + delay | 302 ± 10 | 5549 ± 288 | 18.3 | 90 |

Analysis of the hydrated thickness data for graft polymeric coatings prepared using either continuous, intermittent and intermittent+delay UV irradiation conditions presented in Table 12 show clearly that the coating made using continuous UV irradiation was significantly thinner than the two coatings prepared using intermittent UV irradiation conditions. Furthermore the coating made using intermittent+delay UV irradiation was significantly thicker than the coating made with intermittent UV irradiation.

Analysis of the swelling ratio of the three coatings (see Table 13) indicated that the coatings made with intermittent UV irradiation were able to swell much more that the coating made using continuous UV irradiation when hydrated with PBS solution. This is most likely due to the degree of cross-linking within the coatings. Continuous UV irradiation will lead to four processes; (i) free radical formation, (ii) chain scission, (iii) cross-linking reactions and (iv) polymer chain growth. These four processes will also occur for graft polymeric coatings prepared using intermittent UV irradiation but the relative balance of the four processes will most likely be different. Assuming that the free radical formation is equal in both cases, intermittent UV irradiation should lead to more polymer growth when the sample is not being irradiated with UV than in the continuous case and less cross-linking within the coatings. This hypothesis is borne out by both the dry and hydrated thickness which were both greater in the case of samples prepared with intermittent UV irradiation. The reduced swelling ratio obtained for the samples prepared using continuous UV irradiation suggest that there was a higher degree of cross-linking within the coatings. Thicker coatings such as those prepared using intermittent UV irradiation, with an equal degree of cross-linking would have a very similar swelling ratio. The influence of additional time with no UV irradiation (intermittent+delay) was to increase the polymeric coating graft layer thickness, again suggesting that when the sample was not being irradiated, more polymer growth was occurring than for shorter non-irradiation times (intermittent) and much more polymer chain growth was occurring than in the continuous UV irradiation condition.

Also reported in Table 12 are the modulus values for the three coating conditions obtained from analysis of the AFM direct interaction force data and by fitting of the force curves with model data generated using Hertz theory. Here it is clear that the coatings produced using the two intermittent conditions are slightly softer than those prepared using continuous irradiation. This data supports the hypothesis that there was less cross-linking within the coatings made using intermittent UV irradiation.

### Example 12

### Cellular Response to Different Coating Architectures

### Part A: Formation of Initiator Free UV Graft Copolymer Coatings Using Intermittent Exposure of UV Radiation

As per Example 1, 10% (w/v) aqueous solutions of different molar ratios (0-100%) of the monomers acrylic acid (AA) and acrylamide (AAM) were degassed by purging with nitrogen for more than 15 min in a glove box (oxygen concentration <0.1%). The solutions prepared in this way were then transferred into the wells of 96 well tissue culture polystyrene (TCPS) plates (Nunclon™ Δ, Nunc). The volume of monomer solution added to each well was 0.07 cm³. While still in the glove box, plates containing the monomer solutions described above were then vacuum sealed into polymer bags (Sunbeam FoodSaver) and removed from the glove box. The plates were then passed under a UV lamp (Fusion Systems FS300s, 9 mm D-bulb) 35 times on a conveyor belt (Fusion Systems LC6B Benchtop Conveyor) at a speed of approximately 1.8 m/min. After each pass the plate was rotated 180 degrees to enable more uniform UV irradiation. The plates were then thoroughly washed with running Milli-Q™ water followed by incubation in a large volume of Milli-Q™ water over 72 hours, with daily water changes, at room temperature to remove any remaining monomer or non-covalently bound polymer. Finally the multiwell plate samples were air dried.

### Part B: Formation of UV Graft Copolymer Coatings Based on Macro-Initiators

96 well tissue culture polystyrene plates (Nunclon™ Δ, Nunc) were introduced into a radio frequency glow discharge plasma reactor described elsewhere [Griesser H J., Vacuum 39 (1989) 485]. Plates were placed onto a rectangular copper electrode having the same dimensions as the base of the multiwell plate. Deposition of an allylamine plasma polymer (ALAPP) thin film was then carried out for 25 s at a power of 20 W, a frequency of 200 kHz and an initial monomer pressure of 0.33 mbar.

Subsequently, the in-house synthesised macro-initiator poly(acrylic acid-co-diethyl-dithiocarbamic acid 4-vinyl-benzyl ester) (PI) described elsewhere [L. Meagher, H. Thissen, P. Pasic, R. A. Evans, G. Johnson, WO2008019450-A1] was covalently immobilised on the amine functionalised multiwell plate surface by incubation with a mixture of 90% (v/v) DMF (Merck) and 10% (v/v) Milli-Q™ water containing 3.8 mg/mL N-(3-dimethylaminopropyl)-N′-ethylcarbodiimide hydrochloride (EDC) (Sigma) at room temperature over 2 hours at a PI concentration of 1% (w/v). Plates were then washed 3 times with a mixture of 90% (v/v) DMF (Merck) and 10% (v/v) Milli-Q™ water and 3 times with Milli-Q™ before drying in air.

10% (w/v) aqueous solutions containing different molar ratios (0-100%) of acrylic acid (AA) and acrylamide (AAM) monomers were degassed by purging with nitrogen for more than 15 min in a glove box (oxygen concentration <0.1%). The solutions prepared in this way were then transferred into the wells of PI modified ALAPP treated 96 well tissue culture polystyrene plates. The volume of monomer solution added to each well was 0.20 cm³. While still in the glove box, plates containing the monomer solutions described above were then vacuum sealed into polymer bags (Sunbeam FoodSaver) and removed from the glove box. The plates were then placed under a UV lamp (Spectroline, model XX-15A) and irradiated continuously at an intensity of 10 mW/cm² in a custom-built box for 6 hours to achieve polymerisation. The plates were then thoroughly washed at least 3 times with Milli-Q™ water followed by incubation in a large volume of Milli-Q™ water over 72 hours at room temperature to remove any remaining monomer or non-covalently bound polymer. Finally the multiwell plate samples were air dried.

### Part C: Characterisation of Coating Composition and the Cellular Response

X-ray photoelectron spectroscopy (XPS) analysis was carried out on homo- and copolymer coatings prepared using AA and AAM solutions on TCPS (Part A) or TCPS-ALAPP-PI (Part B) substrates, respectively using the two different UV based coating methods. The results obtained are presented in Table 13 and 14. Analysis of the results demonstrated that a coating was successfully grown from the substrates in all cases. The O/C and N/C elemental ratios observed on coatings produced by the initiator-free intermittent UV coating method (Part A) were close to the expected theoretical values for homopolymeric or copolymeric coatings derived from the specified monomer solutions, providing evidence that the molar ratio of AA and AAM in the coatings were similar to the molar ratio in the monomer feed solutions. A clear trend was also observed for both the O/C and N/C ratios. The same trend was observed on coatings prepared by the macro-initiator-based UV method (Part B). However, in the latter case the observed elemental ratios were somewhat different from the expected theoretical values due to the reduced coating thickness achieved with this method, which resulted in coatings that had a dry thickness less than the probe depth of the XPS technique (i.e. some of the data contained a contribution from both the coating and from the substrate).

TABLE 13
Average elemental ratios obtained from XPS analysis of graft homo- and
copolymer coatings prepared on TCPS substrates using AA and AAM
solutions of varying composition. Coatings were formed using
initiator-free intermittent UV graft polymerisation (Part A).

| Mol % AA | O/C | N/C |
|---|---|---|
| 0 | 0.299 | 0.281 |
| 5 | 0.301 | 0.256 |
| 10 | 0.339 | 0.230 |
| 15 | 0.341 | 0.224 |
| 20 | 0.366 | 0.212 |
| 25 | 0.396 | 0.201 |
| 30 | 0.399 | 0.187 |
| 40 | 0.414 | 0.170 |
| 50 | 0.505 | 0.131 |
| 55 | 0.455 | 0.134 |
| 60 | 0.453 | 0.140 |
| 70 | 0.510 | 0.076 |
| 85 | 0.562 | 0.045 |
| 100 | 0.566 | 0.033 |

TABLE 14
Average elemental ratios obtained from XPS analysis of
graft homo- and copolymer coatings prepared on TCPS-
ALAPP-PI substrates using AA and AAM feed solutions
of varying composition. Coatings were formed using
macro-initiator-based UV graft polymerisation (Part B).

| Sample | O/C | N/C |
|---|---|---|
| ALAPP | 0.152 | 0.112 |
| ALAPP-PI | 0.157 | 0.099 |
| 0 mol % AA | 0.240 | 0.241 |
| 5 mol % AA | 0.278 | 0.241 |
| 10 mol % AA | 0.291 | 0.224 |
| 20 mol % AA | 0.333 | 0.203 |
| 50 mol % AA | 0.302 | 0.092 |
| 100 mol % AA | 0.332 | 0.058 |

The attachment of cells to coatings was evaluated using either HeLa cells, human mesenchymal stem cells (hMSC) or L929 mouse fibroblasts. Cell culture experiments were carried out using surface modified 96 well tissue culture polystyrene plates as well as 96 well tissue culture polystyrene control plates (Nunclon™ Δ, Nunc). Samples prepared using the initiator-free intermittent UV coating method were sterilised by gamma irradiation using a dose of 15 kGy (Steritech). Samples prepared using the macro-initiator-based UV coating method were sterilised immediately before cell culture by incubation with a solution of phosphate buffered saline (PBS) containing penicillin and streptomycin at concentrations of 120 and 200 μg/cm³, respectively over 4 hours at room temperature.

HeLa cell attachment was assessed at a seeding density of 2×10⁴ cells/well in fresh Dulbecco's modified Eagle's medium (DMEM)/Hams F12 medium supplemented with 10% foetal bovine serum (FBS), penicillin, streptomycin and glutamine.

Human mesenchymal stem cell (hMSC) attachment was assessed at a seeding density of 7875 cells/well in Mesencult®-XF medium (Stemcell™ Technologies).

L929 cell attachment was assessed at a seeding density of 7875 cells/well in MEM+GlutaMAX™-I medium (Gibco) supplemented with 10% FBS, 1% v/v non-essential amino acids, and 1% v/v Anti-Anti.

For each cell type, plates were incubated for 24 hours at 37° C. in humidified air containing 5% CO₂

In the case of HeLa cells, the quantification of cell attachment was carried out by washing of the wells with 200 μL of culture medium to remove suspended and loosely bound cells after 24 hours incubation. (3-(4,5-dimethylthiazol-2-yl)-2,5-diphenyltetrazolium bromide) (MTT) in DMEM/Hams F12 solution was then added to each well and plates incubated for 4 hours at 37° C. The medium was removed from each well and replaced with DMSO (100 μL/well). Plates were agitated gently to dissolve the stain for 15 minutes on a plate shaker prior to colorimetric measurement of cell viability at a wavelength of 595 nm. The absorbance values measured from the test samples were expressed as a percentage of those measured in tissue culture polystyrene (TCPS) control well.

In the case of hMSC cells, the quantification of cell attachment was carried out by washing of the wells with 200 μL of culture medium to remove suspended and loosely bound cells after 24 hours incubation. 100 μL [3-(4,5-dimethylthiazol-2-yl)-5-(3-carboxymethoxyphenyl)-2-(4-sulfophenyl)-2H-tetrazolium (MTS) in culture medium was then added to each well and plates incubated for 3 hours at 37° C. and 5% CO₂. Results were read with a microplate reader (BioTek) at 490 and 655 nm. The difference between the readings from both wavelengths were obtained and averaged. The data was then normalised by comparison to the reading obtained from the TCPS surface.

In the case of L929 cells, the quantification of cell attachment was carried out by washing of the wells with 200 μL of culture medium to remove suspended and loosely bound cells after 24 hours incubation. 100 μL [3-(4,5-dimethylthiazol-2-yl)-5-(3-carboxymethoxyphenyl)-2-(4-sulfophenyl)-2H-tetrazolium (MTS) in MEM medium (Gibco) containing 10% FBS and minimal essential amino acids was then added to each well and plates incubated for 3 hours at 37° C. and 5% CO₂. Results were read with a microplate reader (BioTek) at wavelengths of 490 and 655 nm. The differences between the readings at both wavelengths were obtained and averaged. The data was then normalised by comparison to the reading obtained from the TCPS surface.

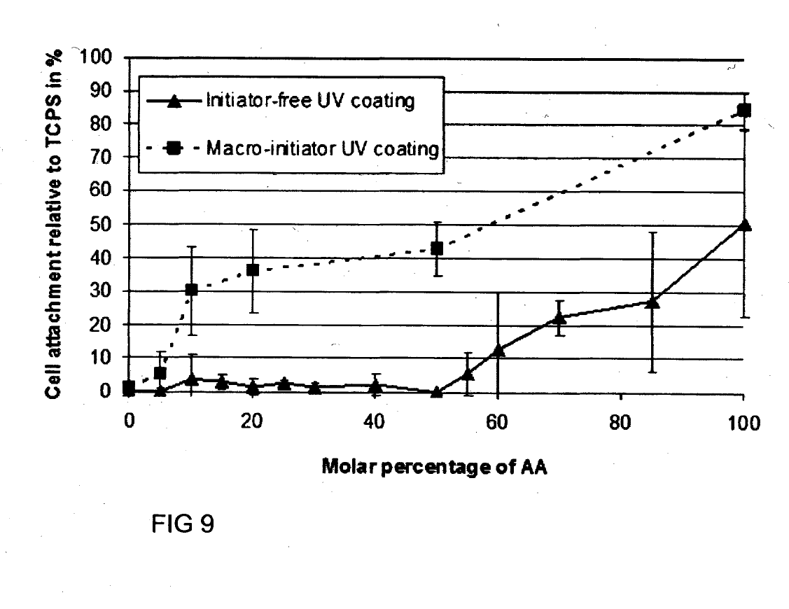

FIG. 9 shows cell attachment results in response to coatings prepared using two different UV methods as a function of the composition of the polymeric coating. The coatings were either homopolymers or copolymers formed from defined molar ratios of acrylic acid (AA) and acrylamide (AAM). For initiator-free UV based coatings produced by intermittent UV exposure (Part A), hMSC attachment was effectively reduced to levels below 10% of the cell attachment obtained on TCPS for molar percentages of AA up to 55%. In comparison, for coatings prepared using the macro-initiator based UV approach (Part B), HeLa cell attachment was only effectively reduced to levels below 10% of the value obtained on TCPS for molar percentages of AA which were below 10%. Lines are drawn to guide the eye (n≧3).

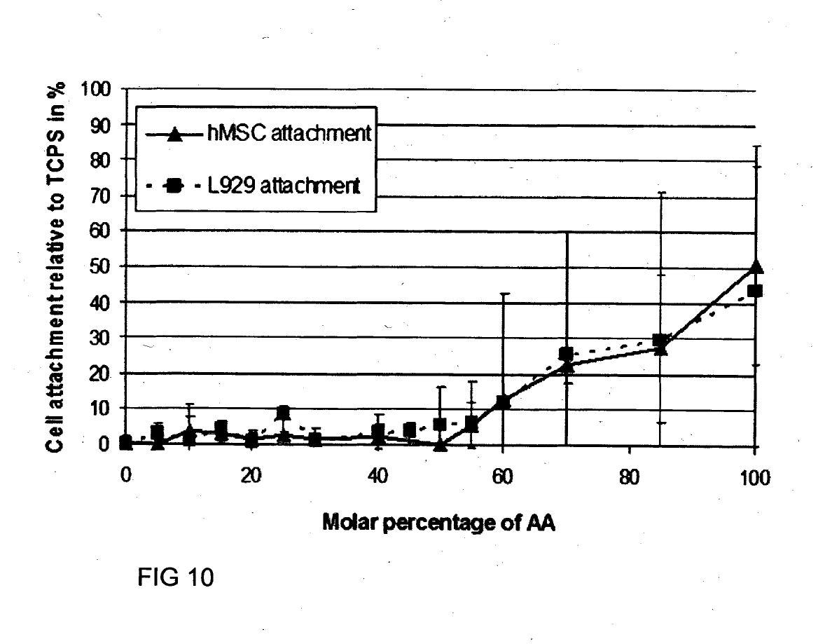

Analysis of the data presented in FIG. 9 clearly suggests that, for the same molar ratio of AA and AAM in copolymer coatings present on a polystyrene substrate surface, different cell attachment responses were observed depending on the coating method. For the initiator-free UV based coatings produced by intermittent UV irradiation (described in Part A), cell attachment can be effectively reduced to levels below 10% of the TCPS value for molar percentages of AA up to 55%. In comparison, for coatings prepared using the macro-initiator based UV approach, cell attachment was only effectively reduced to levels below 10% of the TCPS value for molar percentages of AA below 10%. These results were obtained using human mesenchymal stem cells (hMSC) and HeLa cells, respectively. Furthermore, analysis of the data presented in FIG. 10 clearly shows that similar cell attachment results were obtained with different cell types (with hMSCs and L929 cells) on the same coating. This result was obtained over the whole range of AA/AAM molar compositions for coatings, produced by the initiator-free UV based coating method using intermittent UV. The results clearly demonstrate that similar cell attachment results were achieved. For both cell types, cell attachment was effectively reduced to levels below 10% of TCPS for molar percentages of AA of up to 55%. Lines are drawn to guide the eye.

For comparison, analysis of the data presented in Example 13 shows clearly that for a 40 mol % AA-co-AAM surface prepared using continuous irradiation, the same low L929 adhesion was observed.

FIG. 10 shows the response of different cell types to copolymer coatings based on acrylic acid (AA) and acrylamide (AAM). Coatings were produced by the initiator-free UV based coating method using intermittent UV. Similar cell attachment results were obtained using hMSCs and L929 cells. For both cell types, cell attachment was effectively reduced to levels below 10% of TCPS for molar percentages of AA of up to 55%. Lines are drawn to guide the eye (n≧3).

Overall the data in FIGS. 9 and 10 clearly support the hypothesis that different coating architectures were produced by the two very different UV based polymerisation methods, and that this difference was responsible for the observed differences in the cellular response (i.e. cell attachment). Furthermore, the data clearly demonstrate that coatings produced by the initiator-free UV based coating method were more effective at preventing cell attachment of different cell types over a much wider range of molar ratios of AA and AAM compositions. Due to the fact that cell attachment was enabled by the non-specific adsorption of serum proteins, it can be concluded that the initiator-free UV based coating method is more effective at preventing non-specific serum protein adsorption over a much wider range of AA and AAM molar ratios.

### Example 13

### Polymer Grafting: Continuous Versus Intermittent UV Irradiation

### Part A: Preparation of Coatings

Silicon wafers (Si) were cut into squares of 7×7 mm dimension, ultrasonically cleaned in a 2% (v/v) RBS-35® surfactant, 2% (v/v) ethanol, solution, thoroughly rinsed with Milli-Q™ water, and dried with a high velocity, filtered stream of purified nitrogen gas. Si wafer pieces were further cleaned by UV/ozone treatment in a ProCleaner™ instrument (Bioforce Nanoscience, USA) for 60 minutes immediately before use. Silicon Wafer samples were then were then introduced into a radio frequency glow discharge plasma reactor described elsewhere [Griesser H J., Vacuum 39 (1989) 485]. Samples were placed onto a round lower copper electrode having the same dimensions as the top electrode. Deposition of an allylamine plasma polymer (ALAPP) thin film was then carried out for 25 s at a power of 20 W, a frequency of 200 kHz and an initial monomer pressure of 0.20 mbar. The resulting allylamine coatings (Si-ALAPP) were left in air until further use.

A 7.5% (w/v) aqueous solution containing 40 mol % acrylic acid (AA) and 60 mol % acrylamide (AAM) was prepared in a nitrogen glove box where it was also transferred into PTFE vessels containing Si-ALAPP samples. The volume of monomer solution added to each vessel was 4 mL. While still in the glove box, vessels containing the monomer solutions were then vacuum sealed into polymer bags (Sunbeam FoodSaver) and removed from the glove box. The sealed samples were then exposed to UV radiation generated by a high powered UV lamp (Fusion UV Systems LH6 with 9 mm D-bulb). In normal operation, the sample was positioned under the lamp on a fixed stage and irradiated. A pneumatic shutter was programmed to open and close, such that defined “on” periods of exposure and defined “off” periods of non-exposure could be set. A portable UV meter (EIT UV Power Puck II)) was used to determine the total energy and irradiance reaching the samples under any given settings. Knowing these values, it was then possible to determine equal UV radiation doses across different processing protocols.

The different processing protocols tested are shown in Table 15.

TABLE 15

| Sample | ON^{&} (s) | OFF^{&} (s) | cycles | UVC (J/cm²) |
|---|---|---|---|---|
| Continuous 1 | 15 | n/a | 1 | 0.58 |
| Continuous 2 | 30 | n/a | 1 | 0.99 |
| Continuous 3 | 45 | n/a | 1 | 1.41 |
| Continuous 4 | 60 | n/a | 1 | 1.83 |
| Continuous 5 | 75 | n/a | 1 | 2.25 |
| Continuous 6 | 90 | n/a | 1 | 2.67 |
| Continuous 7 | 120 | n/a | 1 | 3.51 |
| Intermittent (Continuous 6)- | 1 | 20 | 53 | 2.67* |
| 20 OFF-1 | | | | |
| Intermittent (Continuous 6)- | 1.9 | 20 | 35 | 2.67* |
| 20 OFF-2 | | | | |
| Intermittent (Continuous 2)- | 1 | 20 | 19 | 0.99* |
| 20 OFF-1 | | | | |
| Intermittent (Continuous 2)- | 2.9 | 20 | 9 | 0.99* |
| 20 OFF-2 | | | | |
| Intermittent (Continuous 2)- | 4.7 | 20 | 6 | 0.99* |
| 20 OFF-3 | | | | |
| Intermittent (Continuous 2)- | 10 | 20 | 3 | 0.99* |
| 20 OFF-4 | | | | |
| Intermittent (Continuous 2)- | 15 | 20 | 2 | 0.99* |
| 20 OFF-5 | | | | |
| Intermittent (Continuous 2)- | 1 | 1 | 19 | 0.99* |
| 1 OFF-1 | | | | |
| Intermittent (Continuous 2)- | 1 | 10 | 19 | 0.99* |
| 10 OFF-1 | | | | |
| Intermittent (Continuous 2)- | 1 | 30 | 19 | 0.99* |
| 30 OFF-1 | | | | |
| Intermittent (Continuous 2)- | 1 | 60 | 19 | 0.99* |
| 60 OFF-1 | | | | |
| Intermittent (Continuous 2)- | 4.7 | 1 | 6 | 0.99* |
| 1 OFF-3 | | | | |
| Intermittent (Continuous 2)- | 4.7 | 40 | 6 | 0.99* |
| 40 OFF-3 | | | | |
| Intermittent (Continuous 2)- | 4.7 | 60 | 6 | 0.99* |
| 60 OFF-3 | | | | |

*Target values.
^{&}Values represent settings used on the equipment.

After the desired UV radiation exposure, the samples were washed copiously with water and then dried under a filtered purified nitrogen stream prior to analysis.

### Part B: Characterisation of Coatings

XPS analysis of the coatings prepared was carried out and the results obtained are presented in Table 16.

TABLE 16
Atomic percentages and elemental ratios obtained from
XPS analysis of graft polymer coatings formed using
continuous and intermittent processing conditions.

| | O | N | C | | |
|---|---|---|---|---|---|
| Sample | (at. %) | (at. %) | (at. %) | O/C | N/C |
| Si-ALAPP (series 1) | 17.5 | 8.5 | 73.4 | 0.24 | 0.12 |
| Continuous 1 | 26.2 | 9.1 | 63.5 | 0.41 | 0.14 |
| Continuous 2 | 26.4 | 9.2 | 63.5 | 0.42 | 0.14 |
| Continuous 3 | 27.4 | 9.1 | 62.4 | 0.44 | 0.15 |
| Continuous 4 | 27.3 | 9.1 | 62.2 | 0.44 | 0.15 |
| Continuous 5 | 27.4 | 9.0 | 62.3 | 0.44 | 0.14 |
| Continuous 6 | 27.2 | 9.0 | 62.7 | 0.43 | 0.14 |
| Continuous 7 | 25.0 | 10.9 | 63.1 | 0.40 | 0.17 |
| Si-ALAPP (series 2) | 17.6 | 8.5 | 73.0 | 0.24 | 0.12 |
| Intermittent (Continuous 6)- | 27.2 | 9.3 | 62.3 | 0.44 | 0.15 |
| 20 OFF-1 | | | | | |
| Intermittent (Continuous 6)- | 27.6 | 9.3 | 61.8 | 0.45 | 0.15 |
| 20 OFF-2 | | | | | |
| Intermittent (Continuous 2)- | 27.6 | 9.2 | 61.9 | 0.45 | 0.15 |
| 20 OFF-1 | | | | | |
| Intermittent (Continuous 2)- | 27.7 | 9.1 | 61.9 | 0.45 | 0.15 |
| 20 OFF-2 | | | | | |
| Intermittent (Continuous 2)- | 27.5 | 9.2 | 61.9 | 0.44 | 0.15 |
| 20 OFF-3 | | | | | |
| Intermittent (Continuous 2)- | 27.8 | 9.2 | 61.7 | 0.45 | 0.15 |
| 20 OFF-4 | | | | | |
| Intermittent (Continuous 2)- | 27.3 | 9.1 | 62.3 | 0.44 | 0.15 |
| 20 OFF-5 | | | | | |
| Si-ALAPP (series 3) | 16.0 | 12.4 | 71.3 | 0.22 | 0.17 |
| Intermittent (Continuous 2)- | 27.6 | 9.9 | 62.2 | 0.44 | 0.16 |
| 1 OFF-1 | | | | | |
| Intermittent (Continuous 2)- | 27.3 | 9.8 | 62.7 | 0.43 | 0.16 |
| 10 OFF-1 | | | | | |
| Intermittent (Continuous 2)- | 27.3 | 9.5 | 62.9 | 0.43 | 0.15 |
| 30 OFF-1 | | | | | |
| Intermittent (Continuous 2)- | 27.5 | 9.6 | 62.6 | 0.44 | 0.15 |
| 60 OFF-1 | | | | | |
| Intermittent (Continuous 2)- | 27.6 | 9.8 | 62.4 | 0.44 | 0.16 |
| 1 OFF-3 | | | | | |
| Intermittent (Continuous 2)- | 26.6 | 9.9 | 63.3 | 0.42 | 0.16 |
| 40 OFF-3 | | | | | |
| Intermittent (Continuous 2)- | 27.1 | 9.7 | 62.9 | 0.43 | 0.15 |
| 60 OFF-3 | | | | | |

After allylamine plasma polymer thin film deposition (Si-ALAPP), the composition was as expected. After continuous irradiation of the sample in monomer solution, both the O and N atomic percentages increased, consistent with a graft copolymer coating consisting of acrylamide and acrylic acid.

Ellipsometry was used to estimate the thickness of the graft polymer coatings formed (JA Woolam Co, M2000). Phase data were collected at 4 angles (60, 65, 70, and 75 degrees) for 20 seconds at each angle. The data was fitted using a Tauc-Lorentz general oscillator model. The results of ellipsometry experiments are presented in Table 17.

TABLE 17
Thickness data derived from ellipsometric analysis of graft polymer
coatings formed using continuous and intermittent conditions.

| Sample | Thickness* (nm) |
|---|---|
| Si-ALAPP (series 1) | 29.04 |
| Continuous 1 | 15.2 |
| Continuous 2 | 33.81 |
| Continuous 3 | 63.21 |
| Continuous 4 | 95.64 |
| Continuous 5 | 115.93 |
| Continuous 6 | 151.98 |
| Continuous 7 | NM |
| Si-ALAPP (series 2) | 29.52 |
| Intermittent (Continuous 6)-20 OFF-1 | 212.8 |
| Intermittent (Continuous 6)-20 OFF-2 | 196.44 |
| Intermittent (Continuous 2)-20 OFF-1 | 28.68 |
| Intermittent (Continuous 2)-20 OFF-2 | 22.61 |
| Intermittent (Continuous 2)-20 OFF-3 | 37.17 |
| Intermittent (Continuous 2)-20 OFF-4 | 38.33 |
| Intermittent (Continuous 2)-20 OFF-5 | 37.59 |
| Si-ALAPP (series 3) | 32.9 |
| Intermittent (Continuous 2)-1 OFF-1 | 122.09 |
| Intermittent (Continuous 2)-10 OFF-1 | 66.66 |
| Intermittent (Continuous 2)-30 OFF-1 | 96.97 |
| Intermittent (Continuous 2)-60 OFF-1 | 115.53 |
| Intermittent (Continuous 2)-1 OFF-3 | 108.95 |
| Intermittent (Continuous 2)-40 OFF-3 | 134.79 |
| Intermittent (Continuous 2)-60 OFF-3 | 127.83 |

NM = not measured because of insufficient coating quality
*thickness of layer (i.e. not including underlying ALAPP layer)

Sample ‘Continuous 7’ could not be measured. On some replicate samples, the ALAPP had delaminated from the Si substrate owing to elevated temperature and degradation from the long continuous exposure times. On other replicate samples, the coating that had formed was very non-homogenous.

Sample ‘Continuous 6’ appeared to be on the verge of delamination, and in a few areas of some replicate samples delamination occurred. However, the coating thickness was able to be measured. Comparing sample ‘Continuous 6’ and samples ‘Intermittent (Continuous 6)-20 OFF-1’ and ‘Intermittent (Continuous 6)-20 OFF-2’, where all three samples received the same UVC radiation dose of 0.99 J/cm², it is evident that moving from a continuous to an intermitted exposure regime has lead to both a) an ability to prepare coatings without delamination of the ALAPP from the Si, and b) thicker coatings. The increase in thickness obtained in this series of samples with varying intermittent exposure compared to continuous exposure is consistent with the dataset presented in Example 11. In Example 11, intermittent UV exposure resulted in thicker coatings which swelled in PBS to a higher degree and had a lower modulus. It is reasonable to assume that the same trends would be present in the dataset presented here.

One of the clear trends apparent in the dataset is that when the “on” time is varied but the UVC is kept constant, the coating thickness obtained is similar (and generally thicker than the thickness obtained with continuous irradiation). Analysis of this data allows the conclusion that the total UVC dose is very important in determining the thickness of the coating.

There are also some clear trends which may be observed when considering the impact of the “off” time during sample preparation. Example 11 demonstrated that a delay after irradiation resulted in thicker coatings. We concluded that this was due to continued polymerisation after the samples were removed from the UV lamp and that delays before being placed under the UV lamp again allowed for additional polymerisation and a thicker coating. Here we can see that when the “off” time was increased (equivalent to an increased delay time), a greater coating thickness was obtained. For example an “off” time increase of 10 to 60 s (for samples: Intermittent (Continuous 2)-10 OFF-1, Intermittent (Continuous 2-30 OFF-1, and Intermittent (Continuous 2)-60 OFF-1), the thickness increased from 67 to 116 nm. Thus where the “on” time is considered, it is the total UVC exposure which contributes to increased thickness. In the case of the “off” time, the length of the “off” time which contributes to increased thickness.

The culture of L929 fibroblasts with a covalently immobilised, cell adherent cyclic RGDfK peptide were also carried out using a protocol similar to that in previous Examples and compared to controls (no attached peptide). In all cases, the cells attached and spread well on the coatings. No significant differences were noted in the cell number, cell circularity or the area occupied by the cells. Control surfaces prepared by continuous irradiation also resisted cellular adhesion.

### Example 14

### Polymer Grafting: Intermittent UV Irradiation of Increasing Cycles

### Part A: Preparation of Coatings

A 7.5% (w/v) aqueous solution containing 40 mol % acrylic acid (AA) and 60 mol % acrylamide (AAM) was prepared in a nitrogen glove box where it was also transferred into the wells of 48-well tissue culture polystyrene plates containing Si-ALAPP samples as per Example 13. The volume of monomer solution added to each well was 227 uL. While still in the glove box, plates containing the monomer solutions were then vacuum sealed into polymer bags (Sunbeam FoodSaver) and removed from the glove box. The plates were then passed under a UV lamp (Fusion UV Systems LH6, 9 mm D-bulb) for 5, 15, 25, 35, or 45 times on a conveyor belt (Fusion UV Systems DRS 10/12 Conveyor). The wafers were then removed from the monomer solution and thoroughly washed at least 3 times with Milli-Q™ water followed by incubation in a large volume of Milli-Q™ water over 72 hours at room temperature to remove any remaining monomer or non-covalently bound polymer. Finally the samples were air dried.

### Part B: Characterisation of Coatings

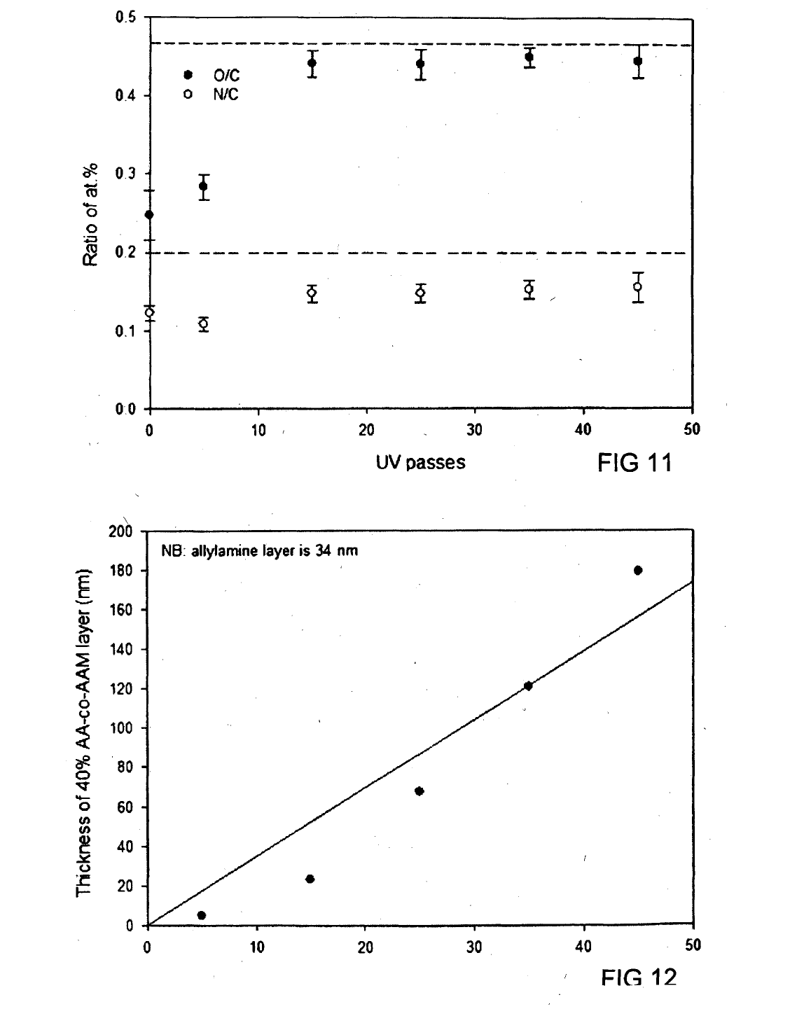

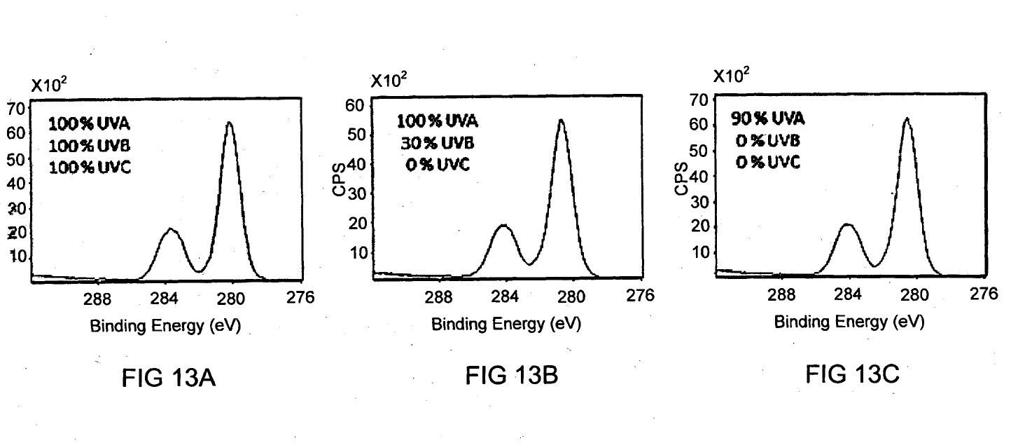

XPS analysis of the coatings prepared was carried out and the results obtained are presented in FIG. 11. As the number of passes increases, the composition determined changed from that of ALAPP towards the theoretical composition of a 40 mol % poly(AA-co-AAM) copolymer, indicating an increasing thickness of the coating to one greater than the XPS sampling depth of approximately 10 nm. The observed deviation from the theoretical composition at higher UV passes may be due to the kinetics of the co-polymerisation.

Ellipsometry was used to estimate the thickness of the graft polymer coatings formed (JA Woolam Co, M2000). Phase data were collected at 4 angles (60, 65, 70, and 75 degrees) for 20 seconds at each angle. The data was fitted using a Tauc-Lorentz general oscillator model. The results of ellipsometry experiments are presented in FIG. 12.

The culture of L929 fibroblasts with a covalently immobilised, cell adherent cyclic RGDfK peptide were also carried out using a protocol similar to that in previous Examples and compared to controls (no attached peptide). In all cases, the cells attached and spread well on the coatings. No significant differences were noted in the cell number, cell circularity or the area occupied by the cells. The one exception was for the sample prepared at 5 UV passes, where cell adhesion was observed presumably because the cell was sensing the underlying ALAPP.

### Example 15

### Polymer Grafting: Effect of Blocking UVB and/or UVC

### Part A: Preparation of Coatings

Silicon wafers (Si) were cut into squares of 7×7 mm dimension, ultrasonically cleaned in a 2% (v/v) RBS-35® surfactant, 2% (v/v) ethanol, solution, thoroughly rinsed with Milli-Q™ water, and dried under purified nitrogen. Si wafer pieces were further cleaned by UV/ozone treatment in a ProCleaner™ instrument (Bioforce Nanoscience, USA) for 60 minutes immediately before use. Silicon Wafer samples were then were then introduced into a radio frequency glow discharge plasma reactor described elsewhere [Griesser H J., Vacuum 39 (1989) 485]. Samples were placed onto a round lower copper electrode having the same dimensions as the top electrode. Deposition of an allylamine plasma polymer (ALAPP) thin film was then carried out for 25 s at a power of 20 W, a frequency of 200 kHz and an initial monomer pressure of 0.20 mbar. The resulting allylamine coatings (Si-ALAPP) were then rinsed in Milli-Q™ water before further use.

A 7.5% (w/v) aqueous solution containing 40 mol % acrylic acid (AA) and 60 mol % acrylamide (AAM) was prepared in a nitrogen glove box where it was also transferred into PTFE vessels containing Si-ALAPP samples. The volume of monomer solution added to each vessel was 4 mL. While still in the glove box, vessels containing the monomer solutions were then vacuum sealed into polymer bags (Sunbeam FoodSaver) and removed from the glove box. In addition, filters were placed inside the polymer bags in some cases in order to attenuate the intensity of UVA, UVB and UVC on the sample surface. The conditions used specifically were: 100% UVA, 100% UVB and 100% UVC; 100% UVA, 30% UVB and 0% UVC; and finally 90% UVA, 0% UVB and 0% UVC. These values were determined using the Power Puck intensity measuring device and various filters. The sealed samples were then removed from the N₂ glove box and exposed to UV radiation generated by a high powered UV lamp (Fusion UV Systems LH6 with 9 mm D-bulb). In normal operation, the sample was positioned under the lamp on a fixed stage. A pneumatic shutter was programmed to open and close, such that defined “on” periods of exposure and defined “off” periods of non-exposure were achieved. A portable UV meter (EIT UV Power Puck II)) was used to determine the total energy and type of radiation reaching the samples under any given setting. In each case, the samples were subjected to 20 cycles of UV exposure where the UV was “on” for 2 s and “off” for 10 s in each cycle.

### Part B: Characterisation of Coatings

XPS results obtained on the coatings are presented in Table 18, where it may be observed that the compositions of all three coatings obtained were very similar, both in terms of atomic percentages, but also in terms of the elemental ratios O/C and N/C. Shown in Figure xxx are representative high resolution C 1s spectra. The shape for the three spectra obtained for each of the samples analysed were also very similar, suggesting that not only the compositions were similar but also the relative proportions of carbon based functional groups. Analysis of the XPS data suggested that grafting occurred in the presence of all three types of UV light investigated, i.e. UVA, UVB and UVC and that all coatings produced had a thickness that was greater than the XPS analysis depth of approximately 10 nm.

TABLE 18
Atomic percentages and elemental ratios obtained from XPS
analysis of graft polymer coatings formed using intermittent
conditions with varying percentages of UVA, UVB and UVC.

| | O | N | C | | |
|---|---|---|---|---|---|
| SAMPLE | (at. %) | (at. %) | (at. %) | O/C | N/C |
| Sample 2 (100% UVA, | 27.7 | 9.0 | 60.9 | 0.45 | 0.15 |
| 100% UVB, 100% UVC) | | | | | |
| Sample 3 (90% UVA, | 26.8 | 9.2 | 63.4 | 0.42 | 0.14 |
| 0% UVB, 0% UVC) | | | | | |
| Sample 4 (100% UVA, | 27.5 | 9.4 | 62.2 | 0.44 | 0.15 |
| 30% UVB, 0% UVC) | | | | | |

Whilst the coatings made with the varying percentages of UVA, UVB and UVC were compositionally similar, the thickness varied in the order 100% UVA, 100% UVB and 100% UVC was thicker than 100% UVA, 30% UVB and 0% UVC was thicker than 90% UVA, 0% UVB and 0% UVC.

### Example 16

### Polymer Grafting: Argon Atmosphere

### Part A: Preparation of Coatings

7.5% (w/v) aqueous solutions of different molar ratios (0-100%) of the monomers acrylic acid (AA) and acrylamide (AAM) were degassed by three cycles of freeze-pump-thaw in a air-tight vessel and then transferred into an argon-filled glove box (oxygen concentration <0.03%). The solutions prepared in this way were then transferred into the wells of 48 well tissue culture polystyrene (TCPS) plates (Nunclon™ Δ, Nunc), with some wells containing Si-ALAPP samples as per Example 13. The volume of monomer solution added to each well was 172 μL (without wafer) and 227 uL (with wafer). While still in the glove box, plates containing the monomer solutions described above were then vacuum sealed into polymer bags (Sunbeam FoodSaver) and removed from the glove box. The plates were then passed under a UV lamp (Fusion UV Systems LH6, 9 mm D-bulb) 40 times on a conveyor belt (Fusion UV Systems DRS 10/12 Conveyor). The plates and wafers were then thoroughly washed with Milli-Q™ water followed by incubation in a large volume of Milli-Q™ water over 72 hours, with daily water changes, at room temperature to remove any remaining monomer or non-covalently bound polymer. Finally the multiwell plate samples and wafers were air dried.

### Part B: Characterisation of Coatings

XPS analysis of the coatings prepared was carried out and the results obtained are presented in Table 19. Analysis of the data obtained from the XPS analysis suggested that the coating compositions were as expected. For example, the atomic percentage of nitrogen in the coating decreased as the mole percentage of AAM monomer in the feed solution was decreased. In parallel with a decrease in the nitrogen content, an increase in the oxygen content was observed as the mole percentage of AA in the monomer feed was increased.

TABLE 19
Atomic percentages and elemental ratios obtained
from XPS analysis of graft polymer coatings formed
using intermittent conditions in an Argon atmosphere.

| | O | N | C | | |
|---|---|---|---|---|---|
| SAMPLE | (at. %) | (at. %) | (at. %) | O/C | N/C |
| Si-ALAPP | 15.6 | 12.6 | 71.8 | 0.22 | 0.18 |
| 0 mol % AA-co-AAM | 16.6 | 19.3 | 64.1 | 0.26 | 0.30 |
| (ie AAM) | | | | | |
| 10 mol % AA-co-AAM | 18.8 | 17.0 | 64.1 | 0.29 | 0.27 |
| 20 mol % AA-co-AAM | 21.2 | 14.7 | 64.1 | 0.33 | 0.23 |
| 30 mol % AA-co-AAM | 23.3 | 12.4 | 64.3 | 0.36 | 0.19 |
| 40 mol % AA-co-AAM | 25.9 | 10.2 | 63.8 | 0.41 | 0.16 |
| 50 mol % AA-co-AAM | 27.5 | 8.8 | 63.8 | 0.43 | 0.14 |
| 60 mol % AA-co-AAM | 27.8 | 8.2 | 63.9 | 0.44 | 0.13 |
| 70 mol % AA-co-AAM | 31.1 | 5.2 | 63.8 | 0.49 | 0.08 |
| 80 mol % AA-co-AAM | 32.4 | 3.7 | 63.8 | 0.51 | 0.06 |
| 90 mol % AA-co-AAM | 34.1 | 2.0 | 63.5 | 0.54 | 0.03 |
| 100 mol % AA-co-AAM | 35.7 | 0.5 | 63.8 | 0.56 | 0.01 |
| (ie AA) | | | | | |

In order to estimate the thickness of the graft polymer coatings formed ellipsometry was used. Phase data were collected at 4 angles (60, 65, 70, and 75 degrees) for 20 seconds each angle. The data was fitted using a Tauc-Lorentz general oscillator model. The results of ellipsometry experiments are presented in Table 20. Analysis of the data obtained indicated that small differences in thickness were apparent which depended on the composition of the monomer feed (i.e. the mole percentage of AA and AAM monomers) and that combinations of AA and AAM above 20 mol % AA gave the thickest coatings while the homopolymer coatings appeared to be slightly thinner.

TABLE 20
Thickness data derived from ellipsometric analysis of graft polymer
coatings formed using intermittent conditions in an Argon atmosphere

| SAMPLE | Thickness* (nm) |
|---|---|
| Si-ALAPP | 27.5 |
| 0 mol % AA-co-AAM (i.e. AAM) | 125.2 |
| 10 mol % AA-co-AAM | 178.9 |
| 20 mol % AA-co-AAM | 200.0 |
| 30 mol % AA-co-AAM | 231.4 |
| 40 mol % AA-co-AAM | 248.0 |
| 50 mol % AA-co-AAM | 221.3 |
| 60 mol % AA-co-AAM | 228.4 |
| 70 mol % AA-co-AAM | 228.2 |
| 80 mol % AA-co-AAM | 243.5 |
| 90 mol % AA-co-AAM | 215.2 |
| 100 mol % AA-co-AAM (ie AA) | 206.6 |

*thickness of layer in question (i.e. not including underlying ALAPP layer)

This data set clearly demonstrates that the coatings can be prepared in the presence of argon as well as nitrogen gas.

### Example 17

### Polymer Grafting Using Monomers from a Wide Range of Chemical Classes

### Part A: Preparation of Coatings

Aqueous solutions or aqueous solutions containing up to 50% DMSO (v/v) were prepared using the monomers listed in Table 21 at concentrations ranging from 0.4 to 1.0 M were prepared in a glove box under a nitrogen atmosphere. Small volumes (40-300 uL) of the solutions were added to the wells of multiwall plates (96 well). At least one row of empty wells was left between rows of wells containing monomer solution to avoid cross-contamination of the polymer coatings. While still in the glove box, plates containing the monomer solutions were then vacuum sealed into polymer bags (Sunbeam FoodSaver) and removed from the glove box. The plates were then passed under a UV lamp (Fusion UV Systems LH6, 9 mm D-bulb) for up to 60 times on a conveyor belt (Fusion UV Systems DRS 10/12 Conveyor). The plates were then thoroughly washed at least 3 times using the solution in which the polymer coating was formed, followed by at least 3 washes with Milli-Q™ water followed by incubation in a large volume of Milli-Q™ water over 72 hours at room temperature to remove any remaining monomer or non-covalently bound polymer. After a further and final washing, the samples were air dried, double bagged and sterilised using gamma irradiation at a dose of 15 KGy prior to characterisation using X-ray photoelectron spectroscopy (XPS).

### Part B: Characterisation of Coatings

XPS analysis of the coatings prepared was carried out and the results obtained are presented in Table 21. Also included for comparison is a typical surface composition of the base substrate, tissue culture treated polystyrene (TCPS). As may be readily observed, the surface composition of coatings formed from the monomers listed in Table 21 were all distinctly different from that of the TCPS substrate, suggesting that a coating was formed in each case. The surface of the TCPS substrate contains only carbon and oxygen. In some cases, the composition was intermediate between the TCPS composition and that of the theoretical composition for the polymer coating of interest. In this case the thickness of the coating formed was less than the XPS sampling depth and the surface composition contained a contribution from the underlying substrate. In many cases the surface composition of the coatings formed on top of the TCPS substrate was very similar to the theoretical composition, indicating that the coating was at least as thick as the XPS sampling depth (10 nm).

TABLE 21
Elemental composition (atomic %) obtained from XPS analysis
of UV graft polymer coatings prepared using monomer solutions
prepared with the monomers listed in the left hand column.

| | Composition (atomic %) | | | | | | | | |
|---|---|---|---|---|---|---|---|---|---|
| Monomer/Sample Name | C | O | N | S | Ca | Na | Cl | K | Si |
| TCPS | 82.0 | 18.0 | | | | | | | |
| N-isopropylacrylamide | 75.6 | 12.2 | 12.2 | | | | | | |
| Methyl methacrylate | 71.6 | 27.6 | | | | | | | 0.8 |
| 2-Carboxyethyl acrylate | 63.0 | 37.0 | | | | | | | |
| 3-Sulfopropyl acrylate, Potassium | 49.2 | 38.5 | 0.6 | 7.4 | 0.9 | 3.5 | | | |
| salt | | | | | | | | | |
| Mono-2-(methacryloyloxy)ethyl | 64.3 | 35.7 | | | | | | | |
| succinate | | | | | | | | | |
| N-(3- | 71.2 | 14.2 | 13.5 | 0.6 | | | 0.6 | | |
| Aminopropyl)methacrylamide | | | | | | | | | |
| hydrochloride | | | | | | | | | |
| 2-Aminoethyl methacrylate | 62.3 | 27.7 | 8.7 | | | | 0.3 | | 0.4 |
| hydrochloride | | | | | | | | | |
| 2-Hydroxyethylmethacrylate | 67.6 | 32.4 | | | | | | | |
| Poly(ethylene glycol) methyl ether | 68.8 | 31.2 | | | | | | | |
| methacrylate [MW 475] | | | | | | | | | |
| Methyl acrylate | 74.8 | 25.1 | 0.1 | | | | | | |
| Poly(propylene glycol) methyl | 72.2 | 27.8 | | | | | | | |
| ether acrylate [MW202] | | | | | | | | | |
| 4-Hydroxybutyl acrylate | 71.8 | 26.2 | 2.0 | | | | | | |
| 2-Methacryloyloxy ethyl | 67.1 | 32.9 | | | | | | | |
| acetoacetate | | | | | | | | | |
| Acrylic Acid | 62.8 | 37.2 | | | | | | | |
| Methacrylic acid | 69.6 | 30.4 | | | | | | | |
| Hydroxyethylacrylate succinate | 60.7 | 35.4 | | | | 3.9 | | | |
| 2-Acrylamido-2-methyl-1- | 55.7 | 27.8 | 6.9 | 6.1 | | 3.6 | | | |
| propanesulfonic acid | | | | | | | | | |
| [3-(Methacryloylamino)propyl] | 73.3 | 13.5 | 10.1 | 0.7 | | | 2.4 | | |
| trimethylammonium chloride | | | | | | | | | |
| [3-(Methacryloyloxy)ethyl]- | 72.0 | 19.2 | 5.8 | 0.5 | | | 2.6 | | |
| trimethylammonium chloride | | | | | | | | | |
| 2-Hydroxyethylacrylate | 64.6 | 35.4 | | | | | | | |
| Poly(ethylene glycol) | 67.5 | 32.5 | | | | | | | |
| methacrylate [MW 360] | | | | | | | | | |
| Poly(ethylene glycol) methyl ether | 70.7 | 29.3 | | | | | | | |
| methacrylate [MW 1100] | | | | | | | | | |
| Poly(ethylene glycol) methyl ether | 70.6 | 29.4 | | | | | | | |
| methacrylate [MW 2080] | | | | | | | | | |
| Poly(ethylene glycol) | 68.3 | 31.8 | | | | | | | |
| methacrylate [MW 526] | | | | | | | | | |
| Acrylamide | 64.0 | 18.6 | 17.5 | | | | | | |
| 1-Vinyl-2-pyrrolidone | 76.9 | 12.9 | 10.2 | | | | | | |
| N,N,-Dimethylacrylamide | 72.7 | 14.1 | 13.2 | | | | | | |
| N-[3-(Dimethylamino)propyl] | 72.4 | 16.3 | 11.0 | 0.4 | | | | | |
| methacrylamide | | | | | | | | | |
| Methacrylamide | 70.5 | 16.7 | 12.8 | | | | | | |
| (2-Dimethylaminoethyl) | 73.2 | 19.2 | 7.3 | | | | | | |
| methacrylate | | | | | | | | | |
| N-(2-Hydroxypropyl) | 70.3 | 19.9 | 9.8 | | | | | | |
| methacrylamide | | | | | | | | | |
| [2- | 63.9 | 26.8 | 4.7 | 4.6 | | | | | |
| (Methacryloyloxy)ethyl]dimethyl- | | | | | | | | | |
| (3-sulfopropyl)ammonium | | | | | | | | | |
| hydroxide | | | | | | | | | |
| [3-(Methacryloylamino)propyl] | 65.1 | 21.9 | 8.7 | 4.3 | | | | | |
| dimethyl(3-sulfopropyl)ammonium | | | | | | | | | |
| hydroxide | | | | | | | | | |
| 4-Acryloylmorpholine | 70.2 | 21.0 | 8.5 | | | | | | 0.5 |
| N-(hydroxymethyl)acrylamide | 62.6 | 24.4 | 12.8 | | | | | | 0.2 |
| N-2-hydroxyethyl acrylamide | 64.5 | 23.6 | 11.3 | | | | | | 0.5 |
| N-Methacryloyl | 64.7 | 27.1 | 7.4 | | | | | | 0.7 |
| tris(hydroxymethyl) | | | | | | | | | |
| aminomethane | | | | | | | | | |
| Sulfopropylmethacrylate | 54.9 | 33.4 | 1.1 | 6.2 | 0.9 | | | 3.5 | |
| Potassium Salt | | | | | | | | | |
| Diacetone acrylamide | 75.3 | 16.5 | 8.2 | | | | | | |
| N,N-Diethyl acrylamide | 78.2 | 11.1 | 10.7 | | | | | | |
| N-Ethyl acrylamide | 70.2 | 16.6 | 13.2 | | | | | | |
| N-(n-propyl)acrylamide | 74.8 | 13.7 | 11.5 | | | | | | |
| Hydroxypropyl acrylate | 67.4 | 31.7 | 0.9 | | | | | | |
| N-tert-Butyl methacrylamide | 75.0 | 18.8 | 6.2 | | | | | | |
| N-tert-Butyl acrylamide | 75.8 | 15.9 | 8.3 | | | | | | |
| N-(n-Butyl)methacrylamide | 77.9 | 13.3 | 8.7 | | | | | | |

1. A process for modifying a polymeric surface, the process comprising;
contacting the polymeric surface with a solution comprising at least one ethylenically unsaturated monomer; and
exposing the polymeric surface in contact with the solution to ultra-violet light to provide a graft-polymer of the monomer coated on the polymeric surface.

2. A process according to claim 1 wherein the step of exposing the polymeric surface comprises intermittently exposing the polymeric surface to ultraviolet light.

3. A process according to claim 2 wherein intermittently exposing the surface comprises at least three periods of UV exposure in the range of from 0.5 seconds to 3 minutes.

4. A process according to claim 2 wherein intermittently exposing the surface comprises at least three periods of UV exposure in the range of from 1 second to 60 seconds.

5. A process according to claim 2 wherein intermittently exposing the surface comprises time periods between exposures in the range of from 5 seconds to 60 minutes.

6. A process according to claim 2 wherein intermittently exposing the surface comprises time periods between exposures in the range of from 10 seconds to 5 minutes.

7. A process according to claim 2 wherein intermittently exposing the surface comprises at least three periods of UV exposure in the range of from 1 second to 15 seconds and time periods between exposures in the range of from 1 second to 60 seconds.

8. A process according to claim 1 wherein the polymeric surface and the solution are each substantially free of initiators.

9. A process according to claim 1 wherein the solution is aqueous.

10. A process according to claim 9 wherein the solution further comprises a water miscible solvent.

11. A process according to claim 1 conducted under an inert gas atmosphere.

12. A process according to claim 1, wherein the polymeric surface is formed of a saturated polymer free of heteroatoms.

13. A process according to claim 1, wherein the polymeric surface is formed from at least one polymer selected from the group consisting of polyethylene, polypropylene, polyisobutylene, ethylene-alphaolefin copolymers, polystyrene, styrene copolymers, poly-isoprene, polybutadiene, polychloroprene rubbers, polyisobutylene rubber, ethylene-propylenediene rubbers and isobutylene-isoprene copolymers.

14. A process according to claim 1, wherein the polymeric surface is formed of polystyrene and the surface is exposed intermittently to ultraviolet light comprising at least three exposures to ultraviolet light for a period in the range of from 1 second to 15 seconds with intervals between exposure in the range of from 1 second to 60 seconds.

15. A process according to claim 1, wherein the at least one ethylenically unsaturated monomer comprises a first monomer comprising a carboxylic acid functional group and a second monomer having low bio fouling properties.

16. A process according to claim 15, wherein the first monomer comprises at least one selected from the group consisting of acrylic acid, methacrylic acid and 2-carboxyethyl acrylate and the second monomer comprises at least one selected from the group consisting of acrylamide, poly(ethylene glycol) (meth)acrylate, methoxy poly(ethylene glycol) (meth)acrylate, N-(2-hydroxypropyl) methacrylamide and 2-methacryloyloxyethyl phosphorylcholine.

17. A process according to claim 15 comprising acrylic acid as a first monomer and acrylamide as a second monomer.

18. A process according to claim 15, wherein the molar ratio of said first monomer to said second monomer in the solution is in the range of from 20:80 to 90:10.

19. A process according to claim 1, wherein the polymeric coating is used in cell culture.

20. A cell culture plate comprising a polymeric surface modified in accordance with the process of claim 1.
